(19) United States
(12) Patent Application Publication
(10) Pub. No.: US 2007/0009895 A1
(43) Pub. Date: Jan. 11, 2007

(54) Method of interaction analysis and interaction analyzer

(76) Inventors: Tadakazu Yamauchi, Chiba (JP); Kouichi Tsuchiya, Chiba (JP); Hideaki Sueoka, Osaka (JP)

Correspondence Address: BIRCH STEWART KOLASCH & BIRCH, PO BOX 747, FALLS CHURCH, VA, 22040-0747, US

(21) Appl. No.: 10/569,951

(22) PCT Filed: Oct. 14, 2004
(86) PCT No.: PCT/JP04/15156

§ 371 Date: Feb. 28, 2006

(30) Foreign Application Priority Data
Oct. 14, 2003 (JP) 2003-35400

(51) Int. Cl. C12Q 1/68; G01N 33/53; C12M 3/00

(52) U.S. Cl. 435/6; 435/7.1; 435/287.2; 977/900

(57) ABSTRACT

A method is provided in which an extremely small amount of a solution containing a substance to be analyzed can be analyzed with high throughput and without any loss of the solution. The method comprises the steps of introducing into a separation channel a first solution containing a substance that is eluted from the separation channel faster and a second solution containing a substance that is eluted from the separation channel more slowly, wherein at least a portion of the first solution is introduced into the separation channel after introducing at least a portion of the second solution thereinto, and detecting a chromatogram of the substances eluted from the separation channel.

### TECHNICAL FIELD

The present invention relates to a method and an apparatus for analyzing interactions which are used for analyzing molecular interactions.

### BACKGROUND ART

To analyze interactions between molecules such as protein-protein, protein-DNA, or protein-low molecular weight compound is indispensable to find functions of biomolecules and effects of pharmaceutical agents. Conventionally many kinds of methods for analyzing molecular interactions have been known, and most of them involve labeling one kind of molecules or immobilizing one kind of molecules to carriers.

On the contrary, recently, the fields of genomics and proteomics have been greatly developed due to the rapid gene-sequencing technology and the rapid protein identification technology with mass spectrometer. Also due to these technologies, the field of chemical genomics has emerged to try to understand the interactions between low molecular weight substances such as pharmaceutical agents and gene expression or protein from a bird's-eye view. In these fields, an approach is needed which allows microanalyses of a number of molecular interactions with high sensitivity and high throughput. Especially, an approach is required which allows multi-analysis of molecular interactions without modifying or immobilizing molecules in the light of faster analyses and avoiding nonspecific effects.

An approach with chromatography is known to analyze interactions of two molecules without modifying or immobilizing the molecules. For example, U.S. Pat. No. 4,762,617 issued to Stevens F. J. discloses a method to determine an interaction between two biopolymers based on an absorbance chromatogram of size-exclusion chromatography. In this method, an absorbance chromatogram of a mixture of two protein molecules is compared to the summation of two absorbance chromatograms of each of the protein molecule to determine if any complexes are formed.

Also, a method for analyzing interactions is reported in Y. Dunayevskiy et al., Rapid Comm. Mass Spectrometry, vol. 11, 1178-1184 (1997), and F. J. Moy et al., Anal. Chem., vol. 73, 571-581 (2001). In this method, a mixture of a protein and a low molecular weight compound is passed through a gel filtration column of spin column type to identify the low molecular weight compound contained in protein fractions with a mass spectrometer. As another example, International Publication WO No. 00/47999 discloses an approach in which a mixture of a target molecule and a ligand is separated by a first size exclusion chromatography to obtain and dissociate complexes of the target molecule and the ligand, and a second size exclusion medium is used to separate the target molecule and the ligand to identify the ligand with a mass spectrometer.

An alternative approach to analyze interactions between two molecules without modifying or immobilizing is reported in JP Paten Publication (Kohyo) No. 2002-508515, in which competitive binding and capillary electrophoresis (CE) are used in combination. In the approach, an interested and detectable target molecule (e.g. proteins), and a known tight binding competition ligand which are tightly bound to the target molecules to change a electrophoresis pattern in CE (e.g. pharmaceutical agents) are mixed with a test sample for capillary electrophoresis to screen the test sample containing a component to bind to the target molecule, by increased peaks caused by the target molecules which were not bound or decreased peaks of the target molecules which formed complexes with the tight binding competition ligand.

JP Paten Publication (Kohyo) No. 2003-502665 discloses an approach to analyze an interaction between a target molecule and a test sample. In the approach, a first plug of a mixture of a target molecule and a test sample, and a second plug of fluorescent labeled tight binding competition ligands, or a first plug of fluorescent labeled tight binding competition ligands, target molecules, and a second plug of a mixture of test samples are continuously introduced into a capillary electrophoresis system to make the second plug pass over the first plug in a capillary tube. An electrophoresis pattern of the competition ligands is obtained at a fluorescent detection section of the system to determine an interaction between the target molecule and the test sample.

In the above mentioned techniques to analyze interactions between molecules with a separation channel such as a chromato-column or an electrophoresis tube, the substances, for example a target molecule and a test sample, to be assayed for the interaction between them are previously mixed before introduction into a separation channel. To assay interactions between a large number of substances, the large number of substances have to be previously mixed each other to prepare sample solutions. Such previous preparation is a complicated work as for a large number of substances, and limits the possibility to decrease the amounts of substances to be analyzed.

For example, when 100 kinds of first substances are combined with 100 kinds of second substances to be assayed, 100×100=10,000 of mixtures are prepared and 10,000 samples are set in an auto injector or the like and are continuously introduced into a separation channel to be analyzed. The more number of substances increases the load of work, and requires a larger auto injector to set the substances.

In addition, the above mentioned techniques have another problem for decreasing the amounts of samples required for analysis. For example, because over amounts of the samples are required to inject accurate amounts of samples into sample tubes with an auto injector, to extract and inject 1 μL of each samples with an auto injector, at least more than several μLs of samples are required for each sample tube. Moreover, several times of the over amounts of the samples are desirably prepared, because the minute samples in the order of several μLs may be evaporated before analysis when a larger number of samples are analyzed, which may result in concentration changes or loss of the samples. So to assay a large number of substances in combination with a large number of substances, from several to dozens of times greater than the amounts of samples are required with respect to an amount an injector actually injects, which is very wasteful. Moreover, in a typical injector, the minimum amount of a sample to be extracted by a syringe and injected into an injection valve is of the order of 1 μL, which needs an approach to effectively achieve combination assays to analyze interactions between molecules with smaller amount of samples.

The above mentioned techniques also have a limitation in decreasing required amounts of samples by reducing the concentrations of samples to be analyzed, because the analyses depend on the sensitivity of detectors. For example, even when a mass spectrometer which provides an advantage to allow unlabeled substances to be identified is used as a detector, no mass chromatogram is obtained with good accuracy with respect to low concentration compounds which are difficult to be ionized. The sample concentrations may be a limit in combining of samples. For example, an approach to analyze substances with high throughput by combining the substances is used, and although the approach has a characteristic advantage to allow the each substance in a mixture to be identified from its mass by a mass spectrometer, when high concentrations of substances are required for analyses, the requirement imposes a restriction on the combinations of the substances. Thus, if a concentration of at least one of two substances to be mapped can be lowered to 1/10 of it for interaction analysis, the combinations of the substances can be increased about tenfold, so that higher throughput will be realized as well as the required amount of the other substance is expected to be lowered to about 1/10 of it. Therefore, in the techniques to analyze interactions between molecules with separation channels such as a chromatography or an electrophoresis tube, an effective means for improving sensitivity for detection is strongly needed.

In view of the above mentioned problems in conventional methods for analyzing interactions, it is therefore an object of the present invention to provide a method and an apparatus for analyzing interactions in which an extremely small amount of a sample can be analyzed with high throughput.

### DISCLOSURE OF THE INVENTION

The present invention to achieve the above object includes:

(1) A method for analyzing interactions, comprising the steps of introducing into a separation channel a first solution comprising a substance to be analyzed that is eluted from the separation channel faster, and a second solution comprising a substance to be analyzed that is eluted from the separation channel more slowly, wherein at least a portion of the first solution is introduced into the separation channel after introducing at least a portion of the second solution thereinto; and detecting a chromatogram of the substances eluted from the separation channel.

(2) The method for analyzing interactions according to (1), characterized by further comprising the step of comparing the detected chromatogram with a chromatogram of the substance comprised in the first solution and/or the substance comprised in the second solution without any interaction with other substances to be analyzed wherein a determination that there exists an interaction between the substance comprised in the first solution and the substance comprised in the second solution is made, when there is a difference between the chromatograms.

(3) The method for analyzing interactions according to (1), characterized in that the separation channel composed of at least one chromatography selected from the group consisting of size exclusion chromatography, ion exchange chromatography, affinity chromatography, adsorption chromatography, hydrophobic chromatography, hydroxyapatite chromatography, metal chelate chromatography, an electrophoresis tube, and an electroosmotic flow tube.

(4) The method for analyzing interactions according to (1), characterized in that the chromatogram is detected by at least one detector selected from the group consisting of a mass spectrometry detector, a spectroscopy detector, a UV detector, a fluorescence detector, a luminescence detector, a refraction detector, and an electrochemical detector.

(5) The method for analyzing interactions according to (1), characterized in that the first solution and/or the second solution comprise a plurality of substances to be analyzed.

(6) The method for analyzing interactions according to (1), characterized in that the chromatogram is a mass chromatogram detected based on the mass of the substance to be analyzed comprised in the first solution and/or the second solution.

(7) The method for analyzing interactions according to (1), characterized in that the first solution and/or the second solution comprise a plurality of substances to be analyzed, and a multiplex chromatogram of the plurality of the substances are detected.

(8) The method for analyzing interactions according to (1), characterized in that the first solution and the second solution are introduced into the separation channel in different amounts.

(9) The method for analyzing interactions according to (1), characterized in that the second solution is introduced into the separation channel in an amount twice or more the amount of the first solution.

(10) The method for analyzing interactions according to (1), characterized in that the step of introducing at least a portion of the first solution into the separation channel after introducing at least a portion of the second solution thereinto comprises introducing a gaseous or liquid spatial sample after the introduction of the second solution and before the introduction of the first solution.

(11) The method for analyzing interactions according to (1), characterized in that the first solution and/or the second solution consist of a plurality of solution samples, and the plurality of solution samples are introduced continuously.

(12) The method for analyzing interactions according to (1), characterized in that the separation channel consists of n stages (n≧2, integer), and a step of introducing a fraction eluted from an (m−1)^{th} stage (2≦m≦n, integer) of the separation channel into an m^{th} stage of the separation channel from m=2 is repeated until m=n, and the step of detecting a chromatogram comprises detecting a chromatogram of a substance to be analyzed eluted from an n^{th} stage of the separation channel.

(13) The method for analyzing interactions according to (12), characterized in that when the fraction eluted from the (m−1)^{th} stage of the separation channel contains the substance comprised in the first solution, the fraction is introduced into the m^{th} stage of the separation channel after the introduction of the second solution, and when the fraction eluted from the (m−1)^{th} stage of the separation channel contains the substance comprised in the second solution, the fraction is introduced into the m^{th} stage of the separation channel before the introduction of the first solution.

(14) The method for analyzing interactions according to (12), characterized in that when the fractions eluted from the (m−1)^{th} stage of the separation channel contain the substance comprised in the first solution, the fractions are introduced into the m^{th} stage of the separation channel during the second solution is introduced into the m^{th} stage of the separation channel at predetermined intervals, and when the fractions eluted from the (m−1)^{th} stage of the separation channel contains the substance comprised in the second solution, the fractions are introduced into the m^{th} stage of the separation channel during the first solution is introduced into the m^{th} stage of the separation channel at predetermined intervals.

(15) The method for analyzing interactions according to claim 1, characterized in that the step of introducing at least a portion of the first solution into the separation channel after introducing at least a portion of the second solution thereinto comprises introducing the first solution and the second solution into an amount of 10 μL or less respectively, preferably in an amount of 3 μL or less respectively.

The present invention can be applied to an apparatus for analyzing interactions to implement the every step comprised in the above method for analyzing interactions. For example, the apparatus for analyzing interactions comprises: a separation device which has a separation channel to separate and elute substances comprised in a solution; a container section which has first solutions comprising the substances that are eluted from the separation channel faster and second solutions comprising the substance that are eluted from the separation channel more slowly; an introduction device to introduce the first solution and second solution from the container section into the separation channel; and a control device to control operation of at least the introduction device. In the apparatus for analyzing interactions, the control device controls the introduction device to introduce at least a portion of the first solution into the separation channel after at least a portion of the second solution is introduced thereinto.

The apparatus for analyzing interactions preferably further comprises a detection device to detect chromatograms of the substances eluted from the separation channel.

Examples of the separation device include at least one of the chromatography selected from the group consisting of a size exclusion chromatography, an ion exchangechromatography, an affinity chromatography, an adsorption chromatography, a hydrophobic chromatography, a hydroxyapatite chromatography, a metal chelate chromatography, an electrophoresis tube device, and an electroosmotic flow tube device.

Examples of the detection device include at least one detector selected from the group consisting of a mass spectrometry detector, a spectroscopy detector, a UV detector, a fluorescence detector, a luminescence detector, a refraction detector, and an electrochemical detector.

The control device may control the introduction device to introduce a gaseous or liquid spatial sample after the introduction of the second solution and before the introduction of the first solution.

The container section may have a plurality of first solutions and/or a plurality of second solutions.

Examples of the separation device include separation channels consisting of n stages (n≧2, integer) so that the control device may repeat a step of introducing fractions eluted from an (m−1)^{th} stage (2≦m≦n, integer) of the separation channel into an m^{th} stage of the separation channel from m=2 until m=n. In this case, when the fractions eluted from the (m−1)^{th} stage of the separation channel contains the substance comprised in the first solution, the control device may control the introduction device to introduce the fractions into the m^{th} stage of the separation channel after the introduction of the second solution, and when the fractions eluted from the (m−1)^{th} stage of the separation channel contains the substance comprised in the second solution, the control device may control the introduction device to introduce the fractions into the m^{th} stage of the separation channel before the introduction of the first solution.

When the fractions eluted from the (m−1)^{th} stage of the separation channel contain the substance comprised in the first solution, the control device may control the introduction device to introduce the fractions into the m^{th} Stage of the separation channel during the second solution is introduced into the m^{th} stage of the separation channel at predetermined intervals, and when the fractions eluted from the (m−1)^{th} stage of the separation channel contain the substance comprised in the second solution, the control device may control the separation device to introduce the fractions into the m^{th} stage of the separation channel during the first solution is introduced into the m^{th} stage of the separation channel at predetermined intervals.

The first solution and the second solution may be introduced into an amount of 10 μL or less respectively, preferably in an amount of 3 μL or less respectively into the separation channel.

According to the present invention, a method and an apparatus for analyzing interactions can be provided in which an extremely small amount of a solution can be analyzed with high throughput without any loss of the solution.

JP Patent Application No. 2003-354000 is the basic application of this application, the specification and/or drawings of which are included herein.

### BRIEF DESCRIPTION OF THE DRAWINGS

FIG. 1 is a block diagram of an apparatus for analyzing interactions according to the present invention;

FIG. 2-1 is a schematic diagram to show a process to introduce a first solution and a second solution into a separation channel, with an apparatus for analyzing interactions according to the present invention;

FIG. 2-2 is a schematic diagram to show a process to introduce a first solution and a second solution into a separation channel, with the apparatus for analyzing interactions according to the present invention;

FIG. 3-1 is a characteristics diagram to show a result of an interaction analysis between the substance (FK506) contained in the first solution and the substance (human FKBP12) contained in the second solution, with a method for analyzing interactions according to the present invention;

FIG. 3-2 is a characteristics diagram to show a result of an interaction analysis between the substance (FK506) contained in the first solution and the substance (human FKBP12) contained in the second solution, with a method for analyzing interactions according to the present invention;

FIG. 3-3 is a characteristics diagram to show a result of an interaction analysis between the substance (FK506) contained in the first solution and the substance (human FKBP12) contained in the second solution, with a method for analyzing interactions according to the present invention;

FIG. 3-4 is a characteristics diagram to show a result of an interaction analysis between the substance (FK506) contained in the first solution and the substance (human FKBP12) contained in the second solution, with a method for analyzing interactions according to the present invention;

FIG. 3-5 is a characteristics diagram to show a result of an interaction analysis between the substance (FK506) contained in the first solution and the substance (human FKBP12) contained in the second solution, with a method for analyzing interactions according to the present invention;

FIG. 3-6 is a characteristics diagram to show a result of an interaction analysis between the substance (FK506) contained in the first solution and the substance (human FKBP12) contained in the second solution, with the method for analyzing interactions according to the present invention;

FIG. 4-1 is a characteristics diagram to show a result of an interaction analysis between the substance (J-8) contained in the first solution and the substance (bovine Calmodulin) contained in the second solution, with the method for analyzing interactions according to the present invention;

FIG. 4-2 is a characteristics diagram to show a result of an interaction analysis between the substance (J-8) contained in the first solution and the substance (bovine Calmodulin) contained in the second solution, with the method for analyzing interactions according to the present invention.

FIG. 4-3 is a characteristics diagram to show a result of an interaction analysis between the substance (J-8) contained in the first solution and the substance (bovine Calmodulin) contained in the second solution, with the method for analyzing interactions according to the present invention;

FIG. 5-1 is a characteristics diagram to show a result of an interaction analysis between a substance contained in the first solution and substances contained in the second solution, with the method for analyzing interactions according to the present invention;

FIG. 5-2 is a characteristics diagram to show a result of an interaction analysis between a substance contained in the first solution and substances contained in the second solution, with the method for analyzing interactions according to the present invention;

FIG. 5-3 is a characteristics diagram to show a result of an interaction analysis between a substance contained in the first solution and substances contained in the second solution, with the method for analyzing interactions according to the present invention;

FIG. 5-4 is a characteristics diagram to show a result of an interaction analysis between a substance contained in the first solution and substances contained in the second solution, with the method for analyzing interactions according to the present invention;

FIG. 6-1 is a characteristics diagram to show a result of an interaction analysis by introducing a first solution consisted of two solution samples and a second solution (containing FK506) into a column, with the method for analyzing interactions according to the present invention;

FIG. 6-2 is a characteristics diagram to show a result of an interaction analysis by introducing a first solution consisted of two solution samples and a second solution (containing FK506) into a column, with the method for analyzing interactions according to the present invention;

FIG. 6-3 is a characteristics diagram to show a result of an interaction analysis by introducing a first solution consisted of two solution samples and a second solution (containing FK506) into a column, with the method for analyzing interactions according to the present invention;

FIG. 6-4 is a characteristics diagram to show a result of an interaction analysis by introducing a first solution consisted of two solution samples and a second solution (containing FK506) into a column, with the method for analyzing interactions according to the present invention;

FIG. 6-5 is a characteristics diagram to show a result of an interaction analysis by introducing a first solution consisted of two solution samples and a second solution (containing FK506) into a column, with the method for analyzing interactions according to the present invention;

FIG. 7-1 is a characteristics diagram to show a result of an interaction analysis by introducing a first solution consisted of two solution samples and a second solution (containing J-8) into a column, with the method for analyzing interactions according to the present invention;

FIG. 7-2 is a characteristics diagram to show a result of an interaction analysis by introducing a first solution consisted of two solution samples and a second solution (containing J-8) into a column, with the method for analyzing interactions according to the present invention;

FIG. 7-3 is a characteristics diagram to show a result of an interaction analysis by introducing a first solution consisted of two solution samples and a second solution (containing J-8) into a column, with the method for analyzing interactions according to the present invention;

FIG. 8-1 is a characteristics diagram to show a result of an interaction analysis by introducing a first solution consisted of three solution samples and a second solution (containing FK506) into a column, with the method for analyzing interactions according to the present invention;

FIG. 8-2 is a characteristics diagram to show a result of an interaction analysis by introducing a first solution consisted of three solution samples and a second solution (containing FK506) into a column, with the method for analyzing interactions according to the present invention;

FIG. 8-3 is a characteristics diagram to show a result of an interaction analysis by introducing a first solution consisted of three solution samples and a second solution (containing FK506) into a column, with the method for analyzing interactions according to the present invention;

FIG. 8-4 is a characteristics diagram to show a result of an interaction analysis by introducing a first solution consisted of three solution samples and a second solution (containing FK506) into a column, with the method for analyzing interactions according to the present invention;

FIG. 8-5 is a characteristics diagram to show a result of an interaction analysis by introducing a first solution consisted of three solution samples and a second solution (containing FK506) into a column, with the method for analyzing interactions according to the present invention;

FIG. 9-1 is a characteristics diagram to show a result of an interaction analysis by introducing a first solution consisted of three solution samples and a second solution (containing Ascomycin) into a column, with the method for analyzing interactions according to the present invention;

FIG. 9-2 is a characteristics diagram to show a result of an interaction analysis by introducing a first solution consisted of three solution samples and a second solution (containing Ascomycin) into a column, with the method for analyzing interactions according to the present invention;

FIG. 9-3 is a characteristics diagram to show a result of an interaction analysis by introducing a first solution consisted of three solution samples and a second solution (containing Ascomycin) into a column, with the method for analyzing interactions according to the present invention;

FIG. 9-4 is a characteristics diagram to show a result of an interaction analysis by introducing a first solution consisted of three solution samples and a second solution (containing Ascomycin) into a column, with the method for analyzing interactions according to the present invention;

FIG. 9-5 is a characteristics diagram to show a result of an interaction analysis by introducing a first solution consisted of three solution samples and a second solution (containing Ascomycin) into a column, with the method for analyzing interactions according to the present invention;

FIG. 10-1 is a characteristics diagram to show a result of an interaction analysis by introducing a first solution separated by a separation channel and introducing a second solution into the separation channel repeatedly at timed intervals, with the method for analyzing interactions according to the present invention;

FIG. 10-2 is a characteristics diagram to show a result of an interaction analysis by introducing a first solution separated by a separation channel and introducing a second solution into the separation channel repeatedly at timed intervals, with the method for analyzing interactions according to the present invention;

FIG. 10-3 is a characteristics diagram to show a result of an interaction analysis by introducing a first solution separated by a separation channel and introducing a second solution into the separation channel repeatedly at timed intervals, with the method for analyzing interactions according to the present invention;

FIG. 10-4 is a characteristics diagram to show a result of an interaction analysis by introducing a first solution separated by a separation channel and introducing a second solution into the separation channel repeatedly at timed intervals, with the method for analyzing interactions according to the present invention;

FIG. 10-5 is a characteristics diagram to show a result of an interaction analysis by introducing a first solution separated by a separation channel and introducing a second solution into the separation channel repeatedly at timed intervals, with the method for analyzing interactions according to the present invention; and

FIG. 10-6 is a characteristics diagram to show a result of an interaction analysis by introducing a first solution separated by a separation channel and introducing a second solution into the separation channel repeatedly at timed intervals, with the method for analyzing interactions according to the present invention.

### BEST MODE FOR CARRYING OUT THE INVENTION

The present invention will now be explained in detail.

1. Method for Analyzing Interactions

The present invention provides a method for analyzing interactions between substances (hereinafter, simply referenced to as “interaction analyzing method”). In the interaction analyzing method, a first solution containing a substance to be analyzed which is eluted faster from a separation channel, and a second solution containing a substance to be analyzed which is eluted more slowly than the substance in the first solution from the separation channel are introduced into the separation channel, and the first solution is introduced after the second solution. Also in the interaction analyzing method, since a portion of the second solution only has to be introduced after the substance in the first solution (which is eluted faster), for example, the second solution may be introduced into the separation channel at predetermined intervals and the first solution may be introduced during the second solution is introduced.

In other words, in this interaction analyzing method, a first solution and a second solution are introduced into a separation channel so that a substance to be analyzed which is eluted faster passes over a substance to be analyzed which is eluted more slowly in the separation channel.

Herein the term “separation channel” is a means to separate and elute a substance depending on its physicochemical properties such as size, ion strength, affinity to certain substances and hydrophobicity, and the like. Examples of a separation channel include a column in a size exclusion chromatography, an ion exchange chromatography, an affinity chromatography, an adsorption chromatography, a hydrophobic chromatography, a hydroxyapatite chromatography, and a metal chelate chromatography. Also examples of a separation channel include a tube in an electrophoresis system, a tube in an electroosmotic flow tube, and the like.

Examples of the substances in first and second solutions include but not limited to any low molecular weight compounds and proteins. For example, when a second solution contains a low molecular weight compound (ligand substance), a first solution may contain a protein (target substance) which is eluted from a separation channel faster than the low molecular weight compound. Since an elution time for a substance depends on the type of a separation channel, the substance in the second solution may be eluted faster than the substance in the first solution in some separation channels.

When the interaction between a protein and a low molecular weight compound is analyzed, because of the large difference between the molecular weights of these two substances, a size exclusion chromatograph is a preferable separation channel. In this case, a first solution containing a substance of a larger molecular weight, and a second solution containing a substance of a smaller molecular weight may be used.

When the difference between charges of two substances is large, an ion exchange chromatograph or an electrophoresis tube is a preferable separation channel. When a substance to be analyzed which may interacts with a carrier filled in a separation channel is analyzed, the elution time should be estimated based on the interaction between the substance and the carrier in order to determine a first solution and a second solution. For example, in a column for size exclusion chromatography with a silica gel chemically bound to diol groups such as glyceropropyl group, in almost all cases, a substance with a large molecular weight such as a protein is eluted faster than a substance with a low molecular weight from such a short separation channel having a length of 30 mm or less, because the low molecular weight compound tends to be dispersed in a gel due to the size exclusion effect and some compounds suffer from a relatively weak adsorption effect between the column carrier and the compound. Thus, when a separation channel is a column for size exclusion chromatography with a silica gel chemically bound to diol groups such as glyceropropyl group, a second solution contains a low molecular weight compound, and a first solution contains a protein. The column for size exclusion chromatography with a silica gel chemically bound to diol groups such as glyceropropyl group is a preferable separation channel, because the column can be used in many applications for interaction analyses between low molecular weight compounds and proteins,

In the interaction analyzing method, first, a second solution is introduced into a separation channel, and after that a first solution is introduced into the separation channel, so that a substance in the first solution passes over a substance in the second solution in the separation channel. This means the substance in the first solution contacts with the substance in the second solution in the separation channel.

When a first solution is introduced into a separation channel after a second solution, the substance in the first solution should be selected to be eluted from the separation channel faster than the substance in the second solution based on the type, length, introduction rate, and the like of the separation channel. In other words, if the substance in the first solution which is introduced later is eluted from the separation channel faster than the substance in the second solution, a gaseous and/or liquid spatial sample may be interposed between the second solution and the first solution.

In the interaction analyzing method, then, a chromatogram of the substances eluted from the separation channel is detected. Specifically, when the substance in the first solution interacts with the substance in the second solution, a chromatogram is detected which is different from chromatograms of the substance in the first solution and/or the substance in the second solution where the substances are introduced into the separation channel separately. If the substance in the first solution does not interact with the substance in the second solution, a chromatogram is detected which is similar to the chromatograms of the substance in the first solution and/or the substance in the second solution where the substances are introduced into the separation channel separately.

Examples of a device to detect chromatogram include but not limited to a mass detector, a spectroscopy detector, a UV detector, a fluorescence detector, a luminescence detector, a refraction detector, and an electrochemical detector.

The first solution and/or the second solution may contain two or more kinds of substances. Thus, in the interaction analyzing method, a first solution containing two or more kinds of substances and a second solution containing two or more kinds of substances may be used, a first solution containing a substance and a second solution containing two or more kinds of substances may be used, or a first solution containing two or more kinds of substances and a second solution containing a substance may be used.

The two or more kinds of substances in a first solution and/or a second solution are the substances to be analyzed. When a solution contains a substance to be analyzed and other foreign substances not to be analyzed, hereinafter, the solution is referenced to contain “a substance”.

When a first solution contains two or more kinds of substances, the substances which are eluted faster than substances in the second solution will be analyzed with respect to their interactions with the substances in the second solution. In other words, if all the substances in a first solution are eluted faster than all the substances in the second solution from a separation channel, the interactions between all the substances in the first solution and all the substances in the second solution will be analyzed.

Even if a first solution and/or a second solution contain two or more kinds of substances, as described above, an interaction between the substances in the first solution and the substances in the second solution can be analyzed by detecting chromatograms of the substances eluted from a separation channel respectively. When a first solution and/or a second solution contain two or more kinds of substances, chromatograms are multiplexed in response to the two or more kinds of substances. All the above listed detection devices may be used to analyze such multiplexed chromatograms.

In particular, a mass spectrometry analyzer is a preferable device to detect multiplexed chromatograms when a second solution contains two or more kinds of proteins and a first solution contains two or more kinds of low molecular weight compounds. A mass spectrometry analyzer is preferable due to its versatility and high throughput because the analyzer can identify each compound based on its mass even when a plurality of compounds are mixed and multiplexed.

A first solution and/or second solution which contain a substance or two or more kinds of substances respectively may consist of a plurality of solution samples. Thus, in the interaction analyzing method, first solutions consisting of a plurality of solution samples and second solutions consisting of a plurality of solution samples may be used, a first solution consisting of a solution sample and second solutions consisting of a plurality of solution samples may be used, or first solutions consisting of a plurality of solution samples and a second solution consisting of a solution sample may be used.

For example, when first solutions consisting of a plurality of solution samples and second solutions consisting of a plurality of solution samples are used, in the interaction analyzing method, first, the second solutions consisting of a plurality of solution samples are all introduced into a separation channel, and after that the first solutions consisting of a plurality of solution samples are introduced into the separation channel. On introducing the first solutions or the second solutions, each solution sample may be introduced into the separation channel with gaseous and/or liquid spatial samples interposing between them or may be introduced continuously in the separation channel.

When a plurality of solution samples are prepared for first solutions and/or second solutions beforehand, the plurality of solution samples may be mixed to a second solution consisting of a solution sample. Alternatively, a plurality of solution samples may be first solutions and/or second solutions without mixing. For example, when the substances contained in a plurality of solution samples have poor solubility or have low concentrations, the plurality of solution samples are preferably first solutions and/or second solutions without mixing. This prevents the substances in the plurality of solution samples from being separated out, and prevents the substances to be analyzed from being of lower concentrations.

In this case again, the interactions between the substances in first solutions and the substances in second solutions can be analyzed by detecting chromatograms of the substances eluted from a separation channel, as described above. When first solutions and/or second solutions consist of a plurality of solution samples, detected chromatograms will be multiplexed in response to the substances in each solution sample. All the above listed detection devices may be used to detect and multiplex such chromatograms. In this way, the substances in solution samples can be introduced continuously in the separation channel without mixing or dilution, resulting in better multiplexed analysis and higher throughput.

As for amount, a first solution and a second solution may be introduced into a separation channel in an equal amount or in different amounts from each other. For example, preferably a second solution is introduced into a more amount than a first solution, such as twice or more. For example, when a second solution contains a low molecular weight compound, and the separation channel contains a size exclusion chromatography, a more amount of second solution than a first solution allows a zone for the low molecular weight compound to be shortened in the separation channel, and the low molecular weight compound to be concentrated because the low molecular weight compound tends to be dispersed in a gel due to the size exclusion effect and some compounds suffer from a relatively weak adsorption effect between the column carrier and the compound. Thus even when a second solution contains a low molecular weight compound in a relatively low concentration, good chromatograms and clear interaction analyses will be obtained. In other words, since a low molecular weight compound in a second solution even in a low concentration can be analyzed clearly, even if a plurality of solution samples are mixed and as a result low molecular weight compounds in each solution sample are in lower concentrations in the mixed solution, the interactions for the compounds can be analyzed clearly.

In the interaction analyzing method, a plurality of separation channels may be used. Assuming that a plurality of separation channels include n separation channels (n≧2, integer) (that is, a separation channel with n stages), in the interaction analyzing method, fractions which are eluted from an (m−1)^{th} stage (2≦m≦n, integer) of the separation channel are introduced into an m^{th} stage of the separation channel, and chromatograms of the fractions eluted from the last separation channel, that is an n^{th} stage in this case, are detected so that, as described above, the interaction between the substance in the first solution and the substance in the second solution is analyzed. The plurality of separation channel may be any combination of those described above such as a chromatography column, an electrophoresis system, an electroosmotic flow tube, and the like as needed.

For example, when a first solution is separated into fractions using the 1^{st} to (n−1)^{th} stages of a separation channel, the fractions are introduced into an n^{th} stage of the separation channel after a second solution is introduced into the n^{th} stage of the separation channel. This means the first solution is prepared in the 1^{st} to (n−1)^{th} stages of a separation channel. The first solution eluted from an (n−1)^{th} stage may contain two or more kinds of substances, or may consist of a plurality of solution samples, as described above.

Also, when a first solution is separated into fractions using the 1^{st} to (n−1)^{th} stages of a separation channel, the fractions may be introduced into an n^{th} stage of the separation channel during the second solution is introduced at predetermined intervals in the n^{th} stage of the separation channel. If the substance contained in a predetermined fraction of the first solution does not interact with the substance in the second solution, the substance in the second solution is to be detected at the predetermined intervals. If the substance contained in a predetermined fraction of the first solution interacts with the substance in second solution, the substance in the second solution are to be detected at offset intervals. Thus, in this case, the detection of the substance in the second solution allows the first solution fractions containing the substance with which the substance in the second solution interacts to be identified.

Meanwhile, for example when a second solution is separated into fractions using the 1^{st} to (n−1)^{th} stages of a separation channel, the fractions are introduced into an n h stage of the separation channel before a first solution is introduced into the n^{th} stage of the separation channel. This means the second solution is prepared in the 1^{st} to (n−1)^{th} stages of a separation channel. The second solution eluted from an (n−1)^{th} stage may contain two or more kinds of substances, or may consist of a plurality of solution samples, as described above.

Also, when a second solution is separated into fractions using the 1^{st} to (n−1)^{th} stages of a separation channel, a first solution may be introduced into an n^{th} stage of the separation channel during the fraction is introduced at predetermined intervals in the n^{th} stage of the separation channel. If the substance contained in a predetermined fraction of the second solution does not interact with the substance in the first solution, the substance in the first solution are to be detected at the predetermined intervals. If the substance contained in a predetermined fraction of the second solution interacts with the substance in the first solution, the substance in the first solution is to be detected at offset intervals. Thus, in this case, the detection of the substance in the first solution allows the second solution fractions containing the substance with which the substance in the first solution interacts to be identified.

As described above, substances the interactions between which are to be analyzed can be previously separated into fractions by using the 1^{st} to (n−1)^{th} stages of a separation channel and separating solutions consisting of complicated components such as cell extracts into first solution fractions or second solution fractions, which enables detail analyses of the interactions between samples consisting of complicated components.

Further separations of the samples into fractions after the interaction analyses are possible, by connecting any combination of those described above such as a chromatography column, an electrophoresis system, an electroosmotic flow tube and the like downstream of the separation channel, which allows the substance which was analyzed by the interaction analyzing method and showed some interactions with other substances to be mapped in detail.

2. Apparatus for Analyzing Interactions

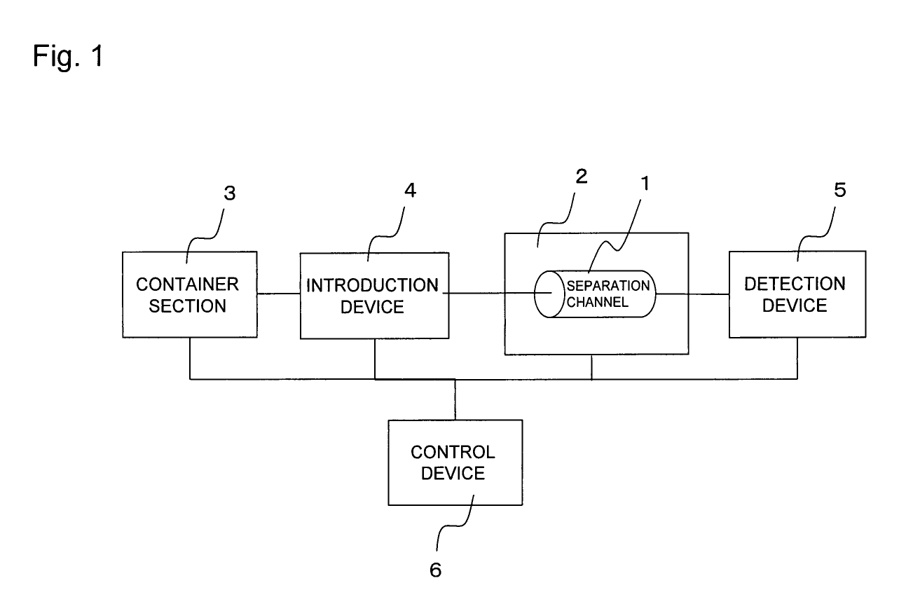

An apparatus for analyzing interactions according to the present invention achieves the method explained in “1. Method for Detecting Interactions”. For example, the apparatus for analyzing interactions comprises, as shown in FIG. 1, a separation device 2 having at least one separation channel 1 to separate and elute substances in solutions, a container section 3 to hold the solutions or the like to be introduced into the separation channel 1, an introduction device 4 to introduce the solutions from the container section 3 to the separation channel, a detection device 5 to detect chromatograms of the substances eluted from the separation device 2, and a control device 6 to control the whole operations of the apparatus. The apparatus for analyzing interactions may be configured so that the separation device 2, the container section 3, the introduction device 4, and the detection device 5 have a control device respectively.

Examples of the separation device 2 include but not limited to a means to separate a substance in a solution introduced into the separation channel 1 depending on its physicochemical properties such as size, ion strength, affinity to certain substances and hydrophobicity, and the like. Examples of the separation device 2 includes at least one chromatography selected from the group consisting of a size exclusion chromatography, an ion exchange chromatography, an affinity chromatography, a normal phase or reversed phase adsorption chromatography, a hydrophobic chromatography, a hydroxyapatite chromatography, a metal chelate chromatography, electrophoresis system, and an electroosmotic flow tube. As used herein, the separation channel 1 refers a column or an electrophoresis tube and an electroosmotic flow tube with which is equipped in each of the above chromatography.

The container section 3 includes a plurality of containers to hold a first solution, a second solution, and an elute to be introduced into to the separation channel respectively. The container section 3 also includes a solution supplier to supply the solutions in the containers in a predetermined amount to the introduction device 4 which will be explained below. Examples of the solution supplier include a syringe and the like. The solution supplier is controlled by the control device 6 to supply a solution in a predetermined amount from a predetermined container to the introduction device 4.

The introduction device 4 includes a so-called auto injector having for example a sample loop to store solutions to be introduced into the separation channel 1, and a pump mechanism to force the solutions stored in the sample loop.

The detection device 5 is arranged on an elution side of the separation channel 1 to detect chromatograms of the substances eluted from the separation channel 1. Examples of the detection device 5 include a mass spectrometry detector, a spectroscopy detector, a UV detector, a fluorescence detector, a luminescence detector, a refraction detector, and an electrochemical detector.

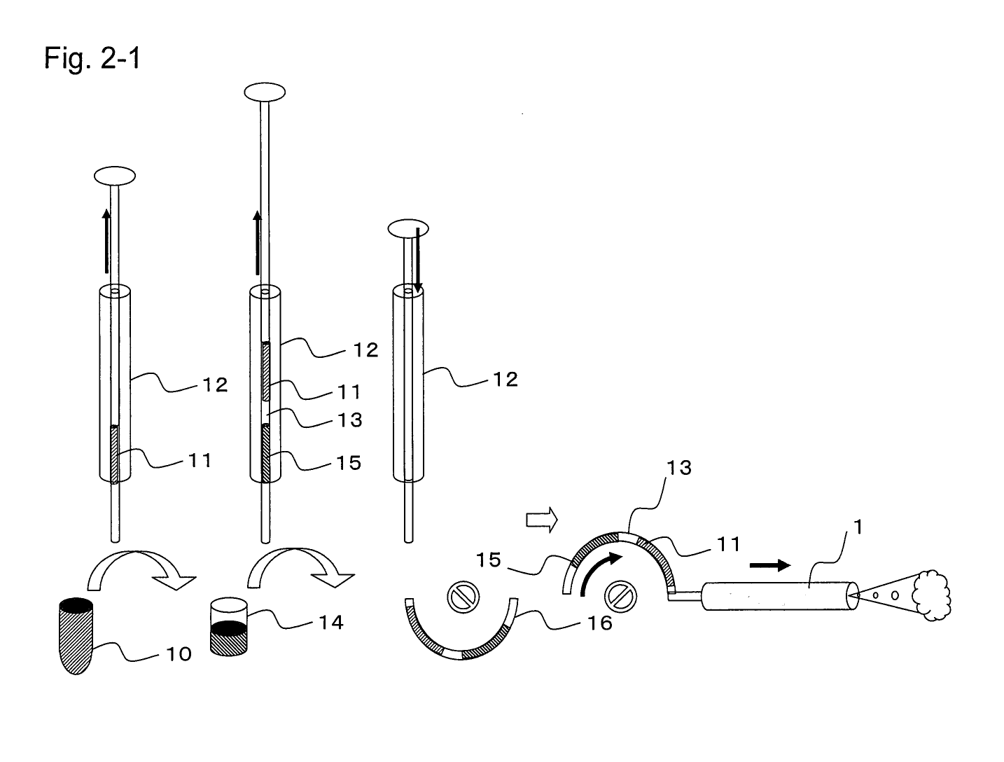

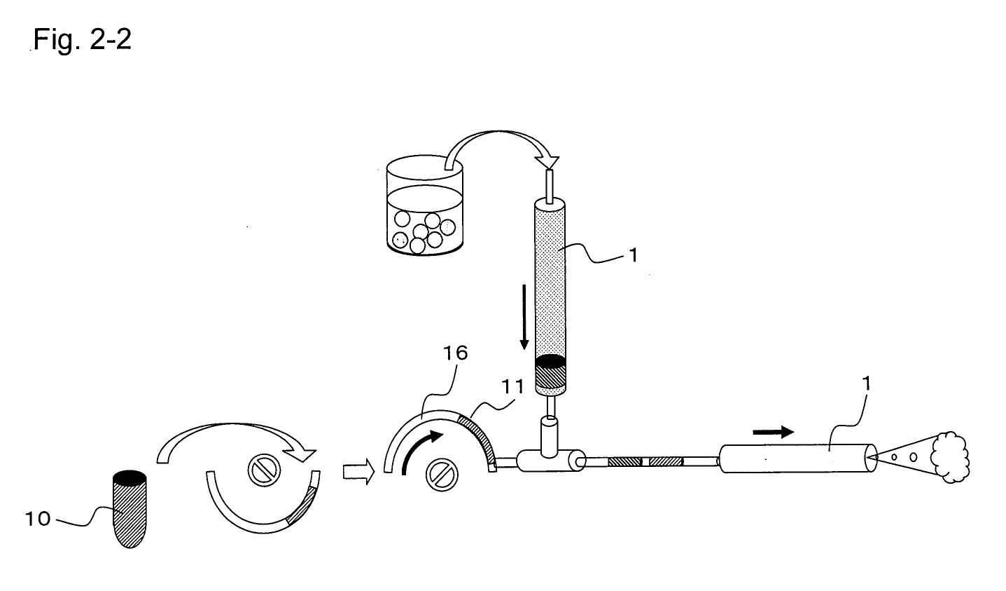

The control device 6 controls the operations of the separation device 2, the container section 3, the introduction device 4, and the detection device 5 to accomplish every step explained in the above “1. Interaction analyzing method”. The control device 6 first controls the introduction device 4 to introduce a second solution and then a first solution in the separation channel 1 from the containers in the container section 3. Specifically, as shown in FIG. 2-1 for example, a second solution 11 held in a first container 10, a predetermined amount of air 13, and a first solution 15 held in a second container 14 are aspirated into a syringe 12 in this order. Then the second solution 11 and the first solution 15 aspirated into the syringe 12 with the air 13 interposed between are supplied to a sample loop 16. The sample loop 16 is pivoted to turn an end of the sample loop 16 on the second solution 11 side to the direction toward the entrance of the separation channel 1. Next, the second solution 11 and the first solution 15 supplied in the sample loop 16 are introduced into this order in the separation channel 1.

As explained in the above “1. Interaction analyzing method”, a second solution and after that a first solution are introduced into the separation channel, so that a substance in the first solution passes over a substance in the second solution in the separation channel. This means the substance in the first solution contacts with the substance in the second solution in the separation channel 1.

Then, the control device 6 controls the detection device 5 to detect chromatograms of the substances eluted from the separation channel 1. Specifically, when the substance in the first solution and the substance in the second solution interact with each other, the detection device 5 detects a chromatogram which is different from chromatograms of the substance in the first solution and/or the substance in the second solution where the substances are introduced into the separation channel separately. If the substance in the first solution does not interact with the substance in the second solution, the detection device 5 detects a chromatogram which is similar to the chromatograms of the substance in the first solution and/or the substance in the second solution where the substances are introduced into the separation channel separately.

According to the apparatus for analyzing interactions, even if a first solution and/or a second solution contain two or more kinds of substances, as described above, an interaction between the substances in the first solution and the substances in the second solution can be analyzed by detecting chromatograms of the substances eluted from a separation channel 1. When a first solution and/or a second solution contain two or more kinds of substances, chromatograms are detected and combined in response to the two or more kinds of substances. All the above listed detection devices 5 may be used to detect and combine such combined chromatograms.

When first solutions and/or second solutions consist of a plurality of solution samples, a plurality of containers hold each solution sample respectively. The control device 6 controls the container section 3 and the introduction device 4 to introduce the plurality of solution samples in a predetermined order into the separation channel 1 so that, in this case, the detection device 5 detects chromatograms and combine them in response to each substance contained in the solution samples. All the above listed detection devices 5 may be used to detect and combine such combined chromatograms. In this way, the substances in the solution samples can be introduced into the separation channel 1 continuously without dilution, resulting in better combined analysis and higher throughput.

As for amount, the control device 6 may control a first solution and a second solution to be introduced into a separation channel 1 in an equal amount or in different amounts from each other. For example, preferably a second solution is introduced into a separation channel 1 in a more amount than a first solution, such as twice or more. For example, when a second solution contains a low molecular weight compound, and the separation channel 1 is a size exclusion chromatograph, a more amount of second solution than a first solution allows a zone for the low molecular weight compound to be shortened at the entrance of the separation channel and the low molecular weight compound to be concentrated because the low molecular weight compound tends to be dispersed in a gel due to the size exclusion effect and some compounds suffer from a relatively weak adsorption effect between a column carrier and low molecular weight compound. Thus even when a second solution contains a low molecular weight compound in a low concentration, good chromatograms and clear interaction analyses will be obtained. In other words, since a low molecular weight compound contained in a second solution at a low concentration can be analyzed clearly, even if a plurality of solution samples are mixed and as a result low molecular weight compounds in each solution sample are in low concentrations relatively, the interactions between the compounds can be analyzed clearly.

In the apparatus for analyzing interactions, the separation device 2 may be configured to include a plurality of the separation channels 1 (e.g., a configuration where n is 2 is shown in FIG. 2-2). Assuming that the plurality of separation channels 1 include n separation channels (n≧2, integer) (that is, a separation channel with n stages), in the apparatus for analyzing interactions, fractions which are eluted from an (m−1)^{th} stage (2≦m≦n, integer) of the separation channel 1 are introduced into an m^{th} stage of the separation channel 1, and chromatograms of the fractions eluted from the last separation channel 1, that is an n^{th} stage of the separation channel 1 in this case, are detected by the detection device 5 so that, as described above, the interaction between the substance in the first solution and the substance in the second solution is analyzed. The plurality of separation channels 1 may be any combination of those described above such as a chromatography column, an electrophoresis tube, an electroosmotic flow tube, and the like as needed.

For example, as shown in FIG. 2-2, when a first solution is separated into fractions using the 1^{st} to (n−1)^{th} stages of the separation channel 1, the fractions are introduced into an n^{th} stage of the separation channel 1 after a second solution is introduced into the n^{th} stage of the separation channel 1. This means the first solution is prepared in the 1^{st} to (n−1)^{th} stages of the separation channel. The first solution eluted from an (n−1)^{th} stage may contain two or more kinds of substances, or may consist of a plurality of solution samples, as described above.

Also, when a first solution is separated into fractions using the 1^{st} to (n−1)^{th} stages of a separation channel, the control device 6 may control the fractions to be introduced into the n^{th} stage during the second solution is introduced into the n^{th} stage at predetermined intervals. If the substance contained in a predetermined fraction of the first solution does not interact with the substance in the second solution, the substance in the second solution are to be detected at the predetermined intervals. If the substance contained in a predetermined fraction of the first solution interacts with the substance in second solution, the substance in the second solution are to be detected at offset intervals. Thus, in this case the detection of the substance in the second solution allows the first solution fractions containing the substance with which the substance in the second solution interacts to be identified.

Meanwhile, for example when a second solution is separated into fractions using the 1^{st} to (n−1)^{th} stages of a separation channel, the fractions are introduced into an n^{th} stage of the separation channel before a first solution is introduced into the n^{th} stage of the separation channel. This means the second solution is prepared in the 1^{st} to (n−1)^{th} stages of a separation channel. The second solution eluted from an (n−1)^{th} stage may contain two or more kinds of substances, or may consist of a plurality of solution samples, as described above.

Also, when a second solution is separated into fractions using the 1^{st} to (n−1)^{th} stages of a separation channel, the control device 6 may control the fractions to be introduced into the n^{th} stage during a first solution is introduced into an n^{th} stage of the separation channel at predetermined intervals. If the substance contained in a predetermined fraction of the second solution does not interact with the substance in the first solution, the substance in the first solution are to be detected at the predetermined intervals. If the substance contained in a predetermined fraction of the second solution interacts with the substance in first solution, the substance in the first solution is to be detected at offset intervals. Thus, in this case, the detection of the substance in the first solution allows the second solution fractions containing the substance with which the substance in the first solution interacts to be identified.

For example, substances the interactions between which are to be analyzed can be previously separated into fractions by using the 1^{st} to (n−1)^{th} stages of a separation channel and separating solutions consisting of complicated components such as cell extracts into first solution fractions or second solution fractions, which enables the detail analyses of interactions between the samples consisting of complicated components.

In the apparatus for analyzing interactions, the separation channel 1 may have a configuration to connect any combination of those described above such as a chromatography column, an electrophoresis tube, an electroosmotic flow tube, and the like downstream thereof, as needed. This connection allows the samples after interaction analyses to be further separated into fractions and allows the substance which was analyzed and showed some interactions with other substances to be mapped in detail.

In the above apparatus for analyzing interactions according to the present invention, for example, when 100 kinds of first solutions are desirably combined with 100 kinds of second solutions to be analyzed, there is no need to prepare 100×100=10,000 of mixtures beforehand, and 100 kinds of first solutions and 100 kinds of second solutions are simply set in the container section 3. In the conventional method and apparatus, 10,000 mixtures should be prepared beforehand, which requires a container section to accommodate the 10,000 mixtures. However, in the apparatus for analyzing interactions according to the present invention, only 200 containers in total should be set, resulting in a space saving of the container section 3.

Also, in the conventional method and apparatus, in order to combine and mix all of a plurality of first solutions and a plurality of second solutions, microamounts of the solutions need to be prepared. To avoid any evaporation of the microamounts of solutions which may result in concentration changes or loss of the samples before analysis, over amounts of the samples should be held in containers. Thus in the conventional method and apparatus, from several to dozens of times greater than the amounts of samples are required for a first solution and a second solution before mixing, which is very wasteful.

To the contrary, in the apparatus for analyzing interactions according to the present invention, since a plurality of first solutions and a plurality of second solutions do not have to be mixed beforehand, even when all of the combination of the solutions are to be analyzed on interactions, only the amounts of solutions for analyses are needed. Thus in the apparatus for analyzing interactions according to the present invention, a plurality of first solutions and a plurality of second solutions can be used without any waste even when all combinations of the first and second solutions are to be analyzed on interactions on a large scale.

Also, in the apparatus for analyzing interactions according to the present invention, since a plurality of first solutions and a plurality of second solutions are not mixed in microamounts, the possibility of any concentration change or loss of the solutions by evaporation before analyses can be significantly reduced. Moreover, because the apparatus for analyzing interactions according to the present invention needs only a mechanism to introduce samples continuously, but no mixing means, the apparatus is especially preferable for microanalysis such as in analytical microchips with micro flow paths. In analytical microchips with micro flow paths which can analyze solutions of less than 1 μL, a plurality of specimens cannot be mixed in micro amounts for combination assay conveniently, but a serial introduction of the specimens can be achieved more easily.

### EXAMPLES

Now the present invention will be explained by way of examples below, but the present invention is not limited to these examples.

### Example 1

Apparatus for Analyzing Interactions

An apparatus for analyzing interactions in this example includes a column TSK super SW2000 for size exclusion chromatography (column size: 1.0 ID×10 mm, 1.0 ID×30 mm or 1.0 ID×100 mm, manufactured by Tosoh Corporation) for a separation channel 1. The apparatus for analyzing interactions also includes an auto injector HTC-PAL (manufactured by CTC Analytics AG) for a container section 3, and a LC pump (Agilent 1100, manufactured by Yokogawa) for an introduction device 4. The apparatus for analyzing interactions also includes an ion trap mass spectrometer LCQ deca XP (ThermoQuest) for a detection device 5.

(1) Configuration of Auto Injector (HTC-PAL)

The auto injector HTC-PAL (CTC Analytics AG) includes a sample loop of 5 μL or 10 μL, a syringe of 10 μL, and sample trays with a cooling unit. A first solution was introduced by 50 μL into a 2 mL screw vial with a conical insert of 100 μL inserted therein. A screw cap with a septum was put on the vial and the vial was placed in a 54 vial rack and the rack was set in one of the sample trays. A second solution was introduced by 40 μL into each well of a 384 well-microplate, and the microplate was covered with an aluminum seal and set in another sample tray. The sample trays were set at the temperature of 10 degrees C.

An analysis method (hereinafter referenced to as Mixing-in-Column Method) in a sequence below was programmed with a macro editor of an auto sampler HTC-PAL (CTC Analytics AG).

Mixing-in-Column Method:

1) Clean syringe inside (solvent 1:50% methanol-water solution);

2) Clean syringe inside (solvent 2: MiliQ water);

3) Aspirate 1 μL of a second substance(s) solution from a well specified by sample sequence;

4) Aspirate 0.5 μL of air;

5) Clean syringe outside (solvent 1:50% methanol-water solution);

6) Clean syringe outside (solvent 2: MiliQ water);

7) Aspirate 1 μL of a first substance(s) solution from a vial specified by sample sequence;

8) Inject the 2.5 μL to injection port;

9) Clean syringe inside (solvent 1:50% methanol-water solution);

10) Clean syringe inside (solvent 2: MiliQ water);

9) Clean injection port (solvent 1:50% methanol-water solution); and

10) Clean injection port (solvent 2: MiliQ water), where the amounts of first solution, second solution, and air are changeable.

An example of sequence controls in an autosampler HTC-PAL is shown below:

syringe:10ul
LC-Inj_with_Separated_P&Chems(2,2,2,2,2,2,0.5,0.5,CStk1-01,1,1,2.5)
[MACRO LC-Inj_with_{—} Separated_P&Chems]
Pre Clean with Solvent 1 ( );1;0;99
Pre Clean with Solvent 2 ( );2;0;99
Post Clean with Solvent 1 ( );2;0;99
Post Clean with Solvent 2 ( );2;0;99
Valve Clean with Solvent 1 ( );2;0;99
Valve Clean with Solvent 2 ( );2;0;99
Pre Air Volume (μl);1;0;SYR.Max Volume
Post Air Volume (μl);1;0;SYR.Max Volume
Protein Rack Position;TRAY
Protein Index ( );1;1;54
Protein Volume (μl);1;0;SYR.Max Volume
Injection Volume (μl);5;0;SYR.Max Volume
WAIT_SYNC_SIG(Start,)
CLEAN_SYR(Wash1,Pre Clean with Solvent 1,,,,,,,)
CLEAN_SYR(Wash2,Pre Clean with Solvent 2,,,,,,,)
GET_SAMPLE(SL.tray,SL.index,SL.volume,Post Air Volume,,,2,,2,,Off,,,)
MOVETO_OBJECT(Wash1,,,)
MOVETO_OBJECT(Wash2,,,)
MOVETO_OBJECT(Wash1,,,)
MOVETO_OBJECT(Wash2,,,)
GET_SAMPLE(Protein Rack Position,Protein Index,Protein Volume,0,,,2,,2,,Off,,,)
MOVETO_OBJECT(LC Vlv1,,,)
WAIT_FOR_DS( )
INJ_SAMPLE(LC Vlv1,Inject,Injected,,Injection Volume,500,,,1,)
CLEAN_SYR(Wash1,Post Clean with Solvent 1,,,,,,,)
CLEAN_INJ(Wash1,LC Vlv1,Valve Clean with Solvent 1,,,,,,,,,)
CLEAN_SYR(Wash2,Post Clean with Solvent 2,,,,,,,)
CLEAN_INJ(Wash2,LC Vlv1,Valve Clean with Solvent 2,,,,,,,,,)
[MACRO METHOD ENTRY]
LOCK_TERMINAL(On,)
CLEANUP(Wash1,Off,Off,On,Off,On,Off,Off,)
[MACRO METHOD EXIT]
CLEANUP(Wash1,Off,Off,Off,On,Off,Off,On,)
LOCK_TERMINAL(Off,)

(2) Configuration of LC Pump (Agilent 1100) and Ion Trap Mass Spectrometer LCQ deca XP

A column TSK super SW2000 for size exclusion chromatography was connected to an outlet port of an injection valve equipped on the auto-injector, and the downstream side of the column was connected to an ESI probe of an ion trap mass spectrometer LCQ deca XP via a Tee connector (PEEK Mixing Tee; GL Sciences Inc.). The LC pump (Agilent 1100) includes a quaternary pump (Q PUMP) having a solution feed line for column equilibrating, and the solution feed line was connected to an inlet port of the injection valve of the autoinjector, and a solution feed line of a binary pump (B pump) for ESJ conditioning was connected to the Tee connector. From the quaternary pump, ammonium acetate solution 10 mM was fed at a rate of 40 μL/min as column equilibrating solution, and from the binary pump (B pump), 0.5% formic acid/methanol solution for measurement in a positive ion mode or 0.5% ammonia/methanol solution for measurement in a negative ion mode was fed at a rate of 10 μL/min as conditioning solution. To stabilize the pH of interaction analyses, a buffer solution such as PIPES buffer solution, ADA buffer solution, HEPES buffer solution, Bis-Tris-hydrochloric buffer solution, or Tris-hydrochloric buffer solution (pH 7.5) was added, as needed.

(3) Measurement

The second solution and the first solution from the wells and vials specified by the sample sequence were automatically introduced continuously with the above Mixing-in-Column method. Thus, as an example, the second solution 1 μL, a spatial sample of air 0.5 μL, and the first solution 1 μL were aspirated into the syringe of the auto injector. Then the 2.5 μL total sample which was aspirated into the syringe was injected into the sample loop, and was introduced into the column of the size exclusion chromatography in the order of second solution-spatial sample-first solution, from the injection port of the injection valve to start to obtain mass chromatograms in a mass spectrometer. After a predetermined period of the measurements for mass chromatograms, a next second solution and a next first solution were aspirated continuously in the same way as described above according to the sample sequence, and a combination assay for interaction analysis was performed. The amounts of the first solution, the second solution, and the air are changeable.

Measurement 1

Interaction Analysis with Mixing-in-Column Method

(Sample Preparation)

First solutions containing a protein were prepared with compositions as follows respectively. Hereinafter, a substance in the first solution is referenced to as a first substance.

First Solution (a);

No first substance (Reference)
10 mM ADA buffer solution (pH 6.5)
100 μM CaCl₂

First Solution (b);
first substance: Bovine Calmodulin
100 μM Bovine Brain Calmodulin (Calbiochem; Code. 208694)
100 μM CaCl₂

First Solution (c);
first substance: Human FKBP12
100 μM Human FKBP12
10 mM ADA buffer solution (pH 6.5)
   Second solutions containing a low molecular weight compound were prepared with compositions as follows respectively. Hereinafter, a substance in the second solution is referenced to as a second substance.

Second Solution (a);
No second substance (Reference)
5% DMSO
50 μM Cyanocobalamin (Reference)

Second Solution (b);
second substance: J-8
100 μM J-8
50 μM Cyanocobalamin (Reference)
5% DMSO

Second Solution (c);
second substance: FK506
50 μM FK506
50 μM Cyanocobalamin (Reference)
5% DMSO
   (Measurement and Result)

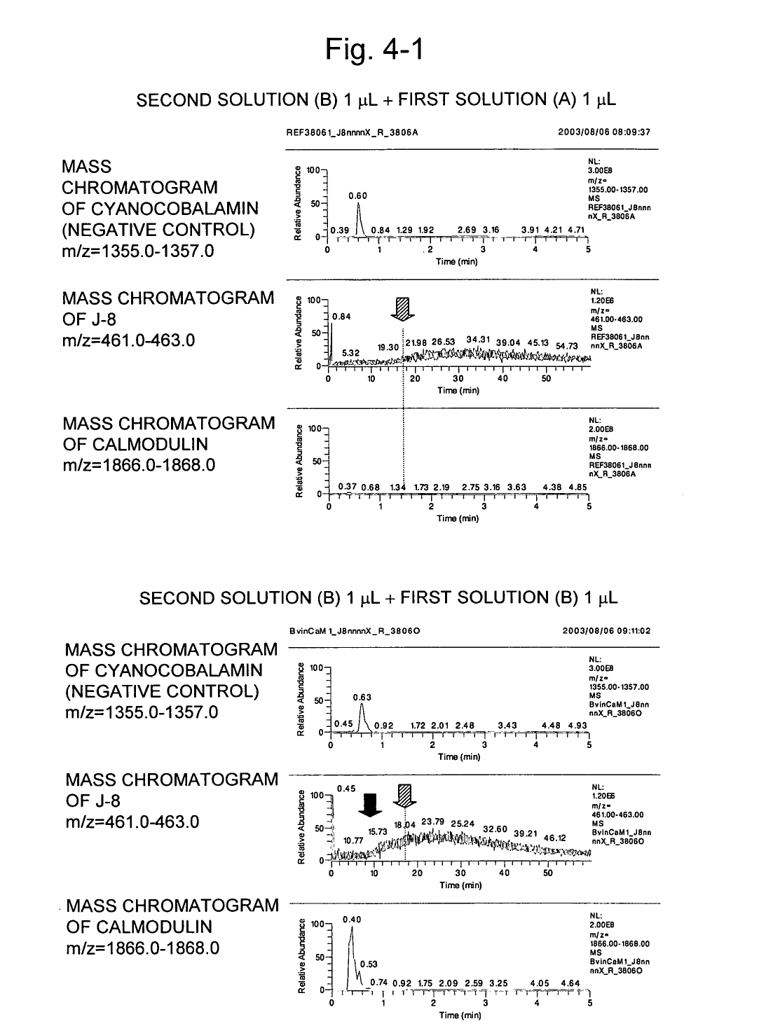

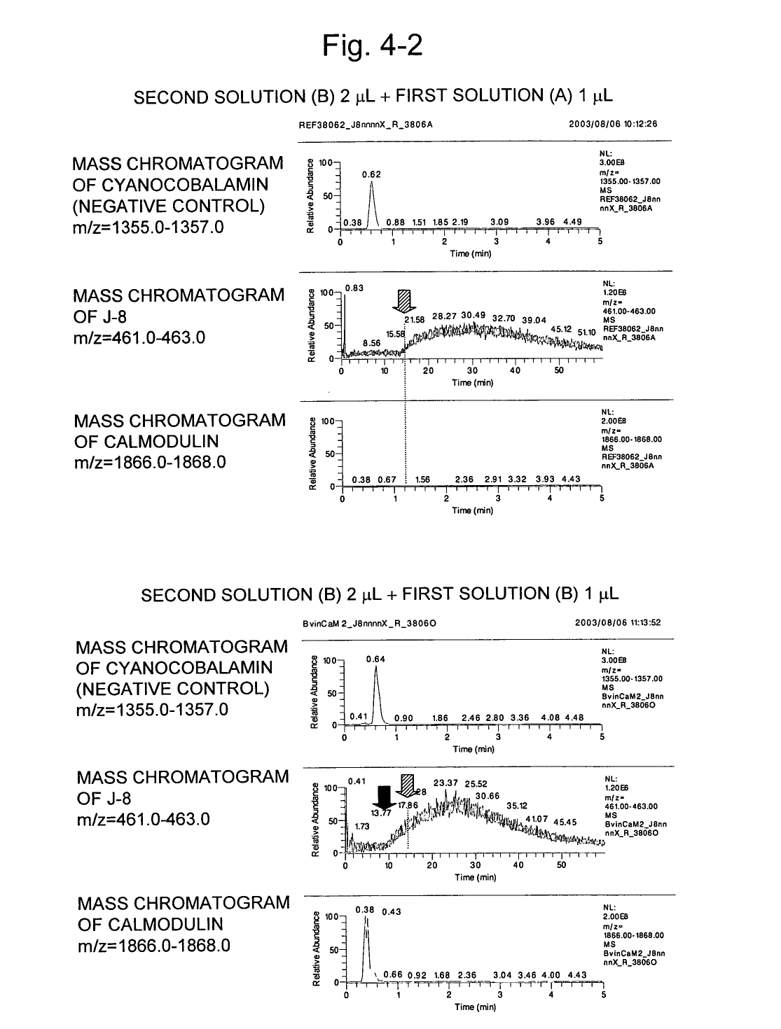

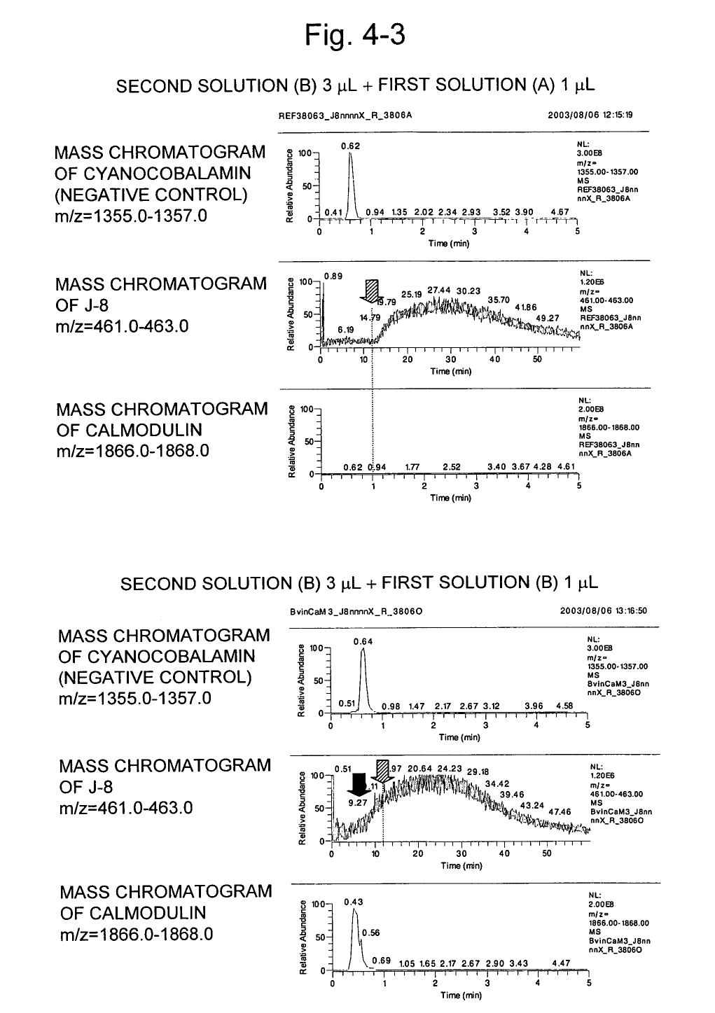

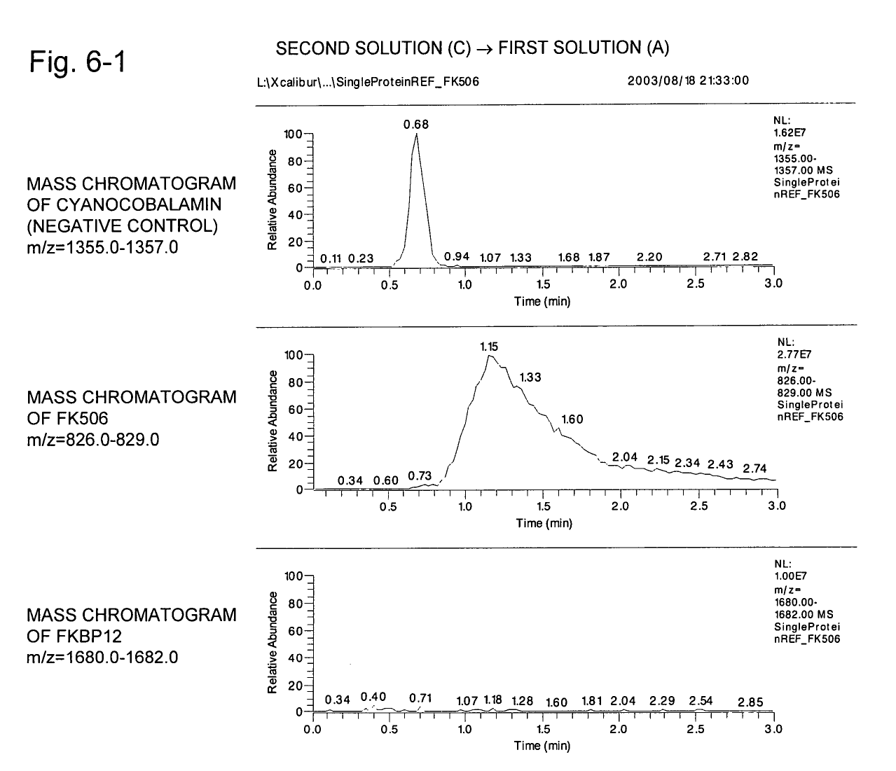

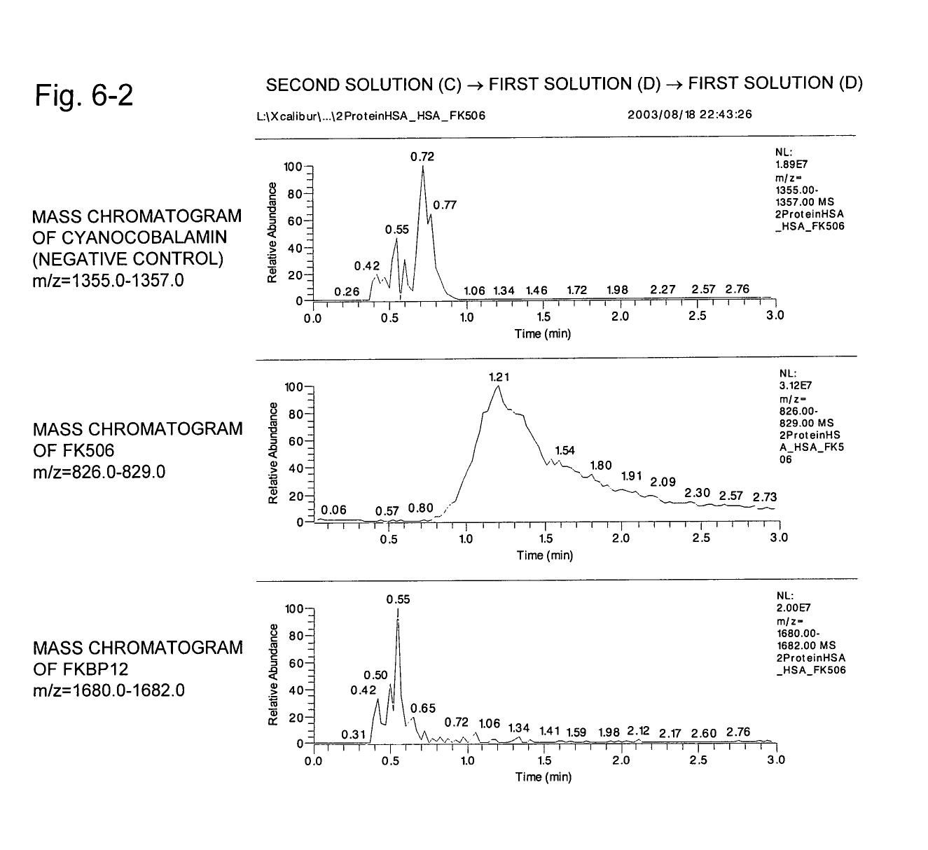

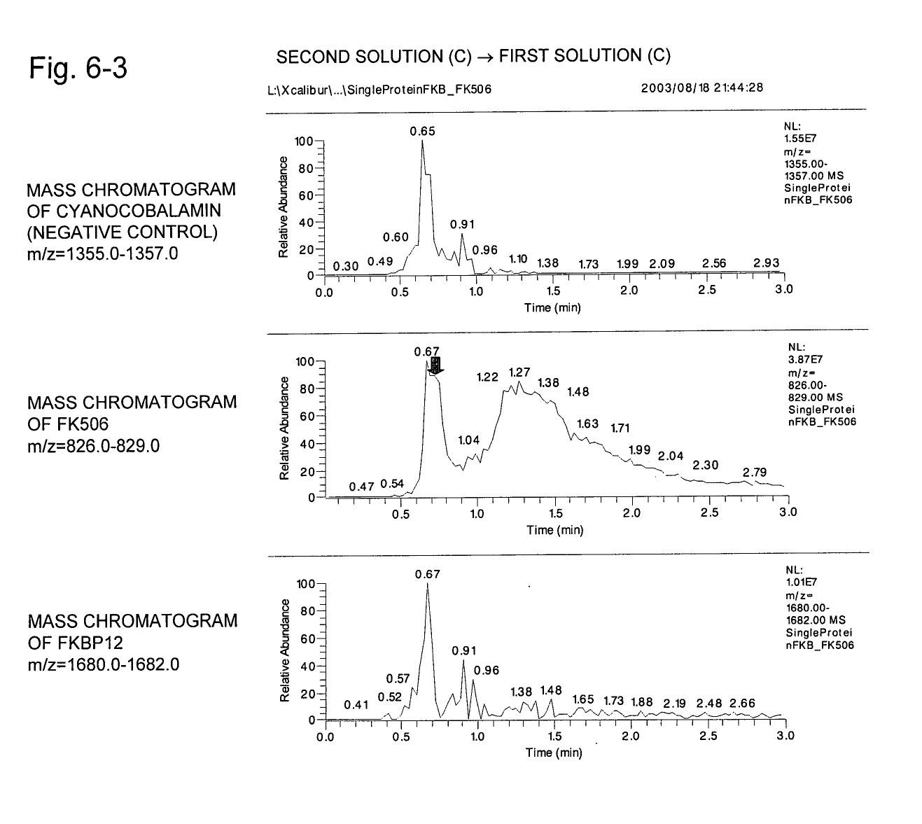

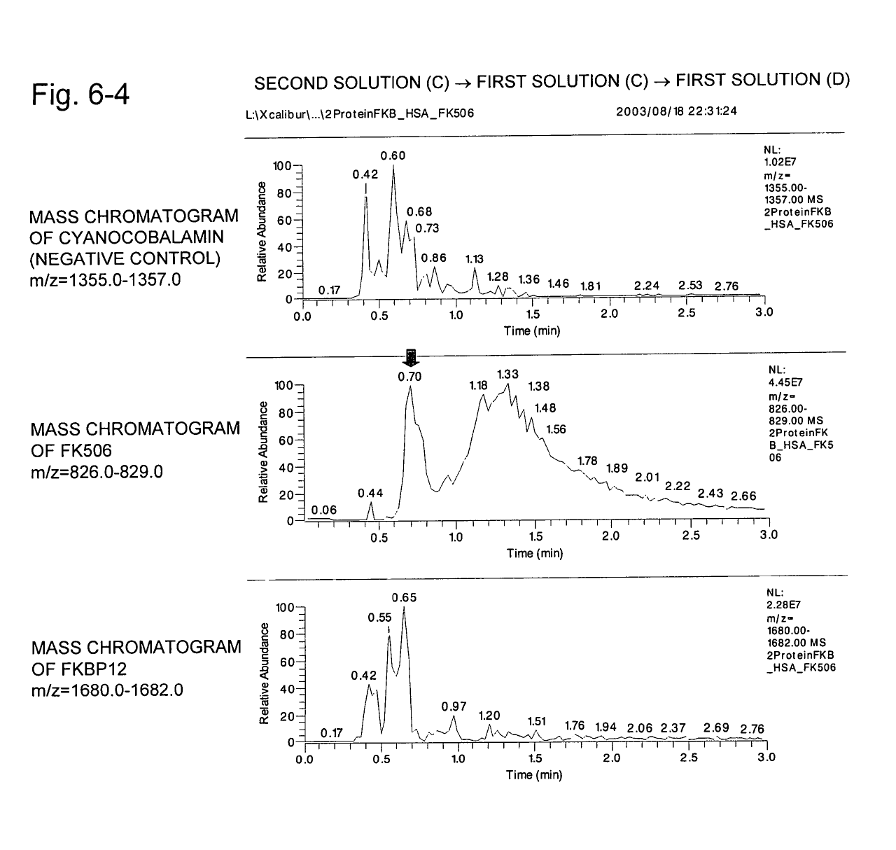

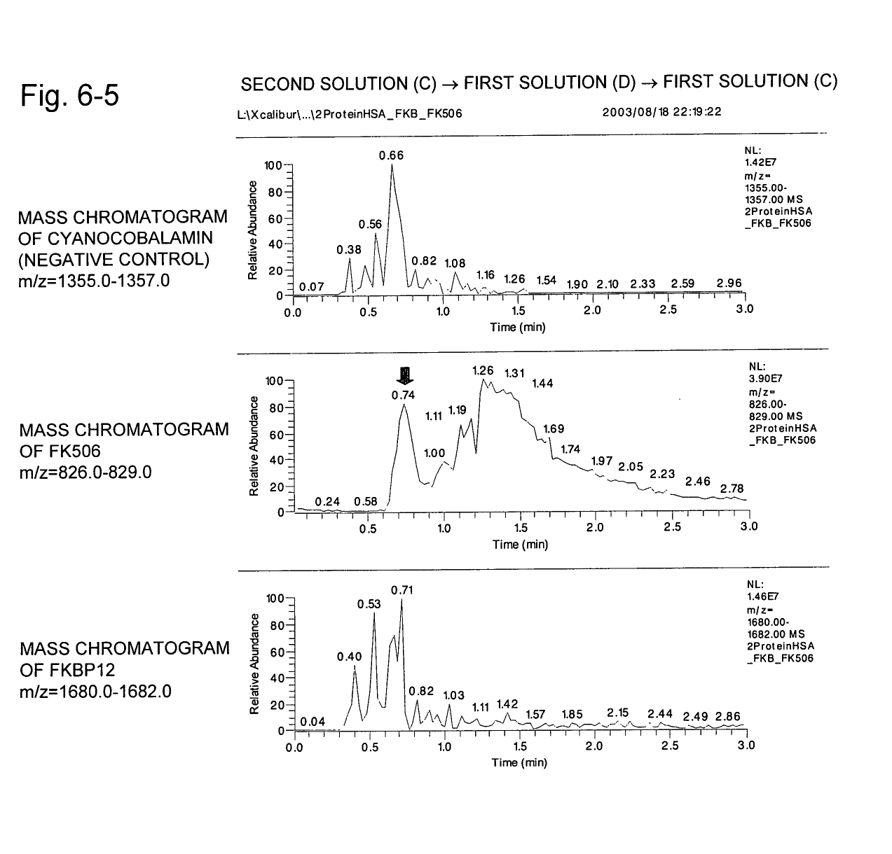

The second solution and the first solution were introduced continuously into the TSK super SW2000 column by the apparatus for analyzing interactions and the Mixing-in-Column method to obtain mass chromatograms for each compound (first substances and second substances). The results are shown in FIGS. 3-1 to 3-6 and FIGS. 4-1 to 4-3.

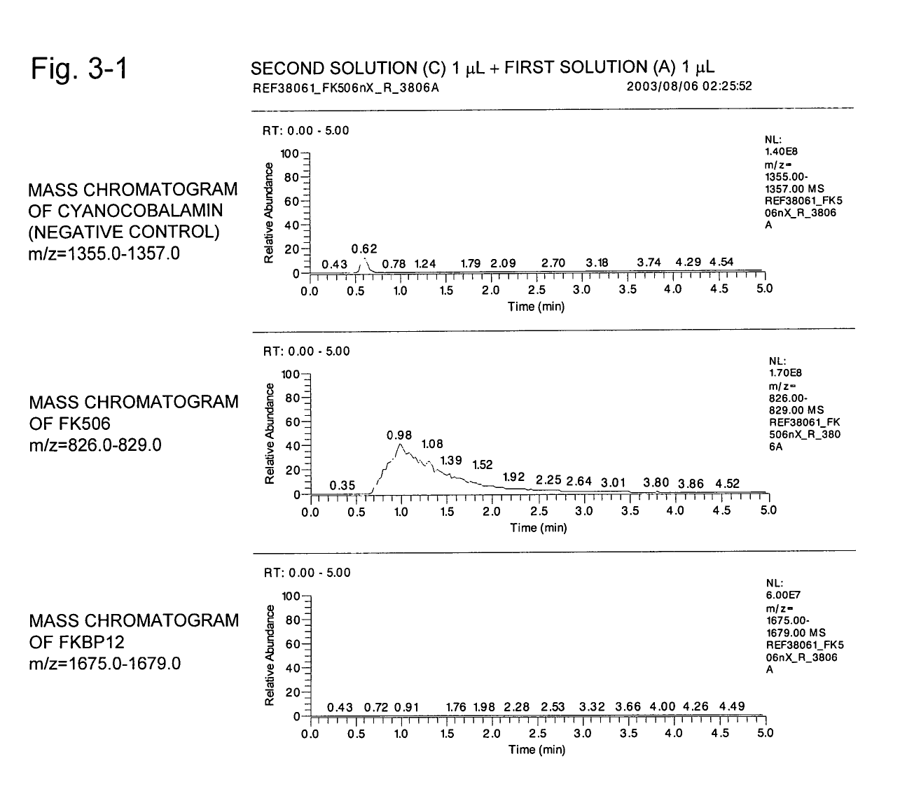

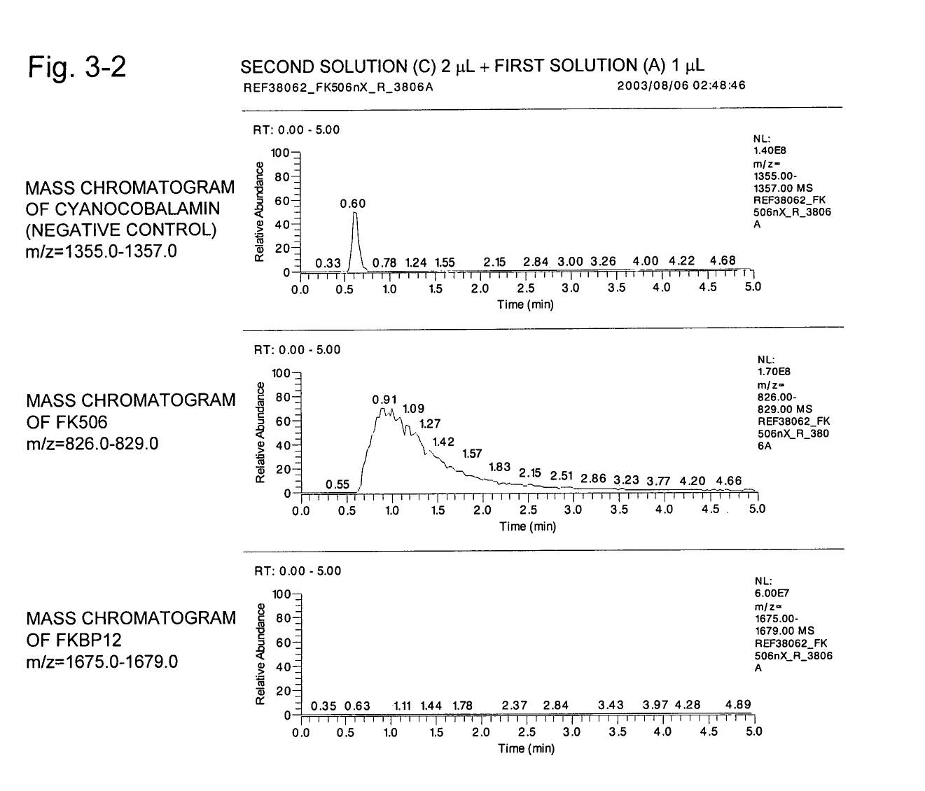

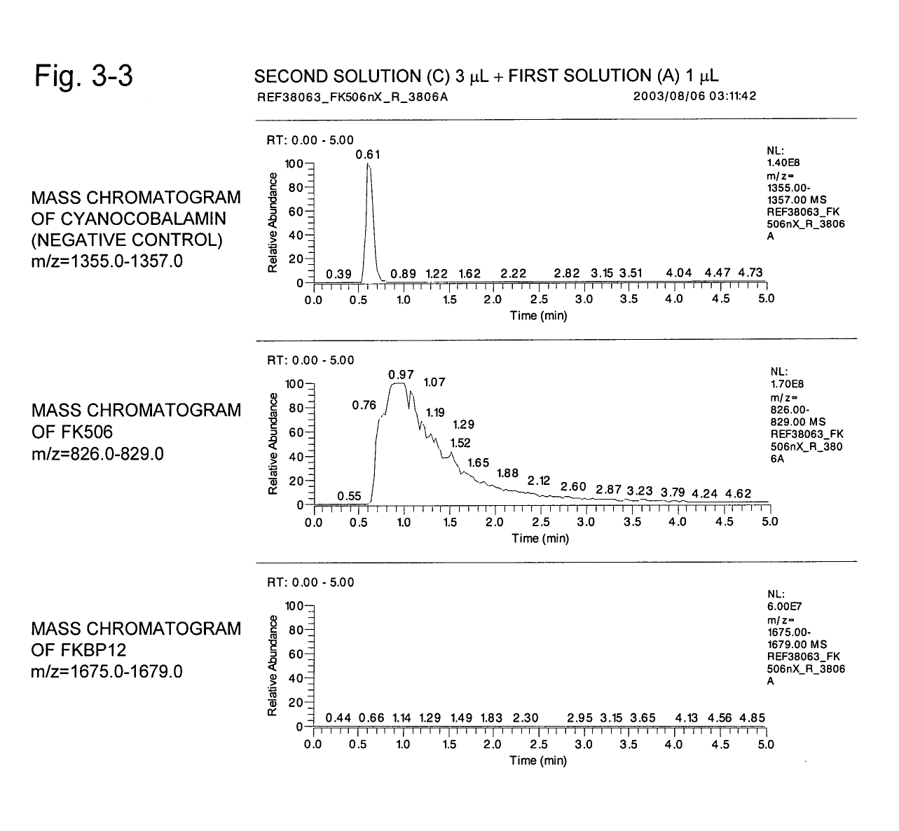

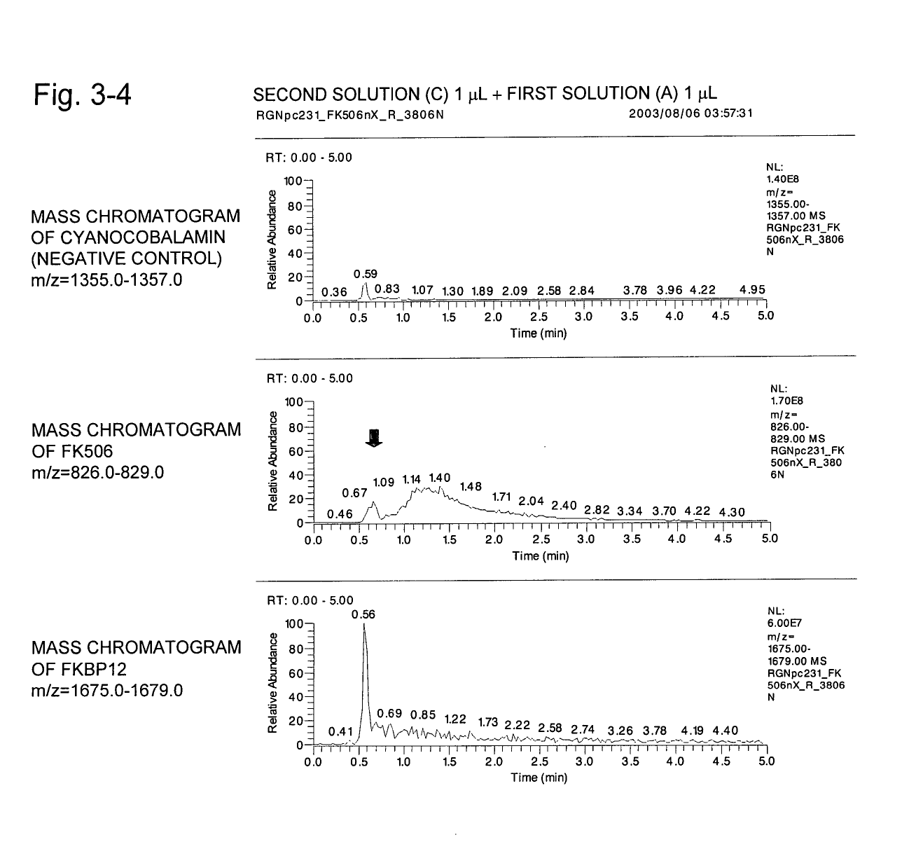

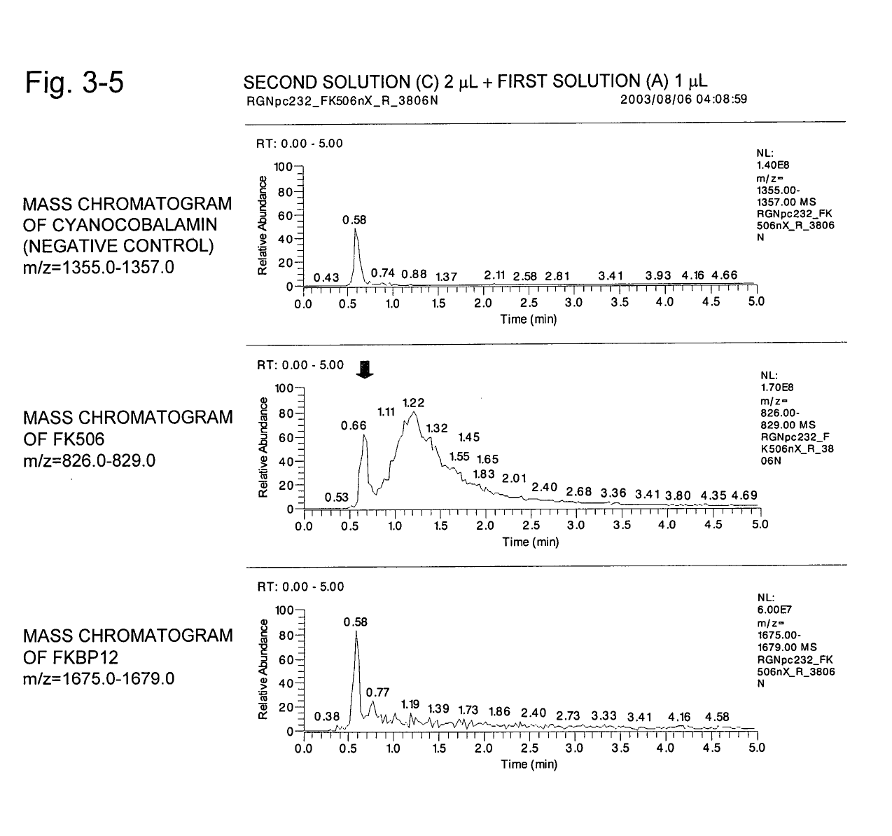

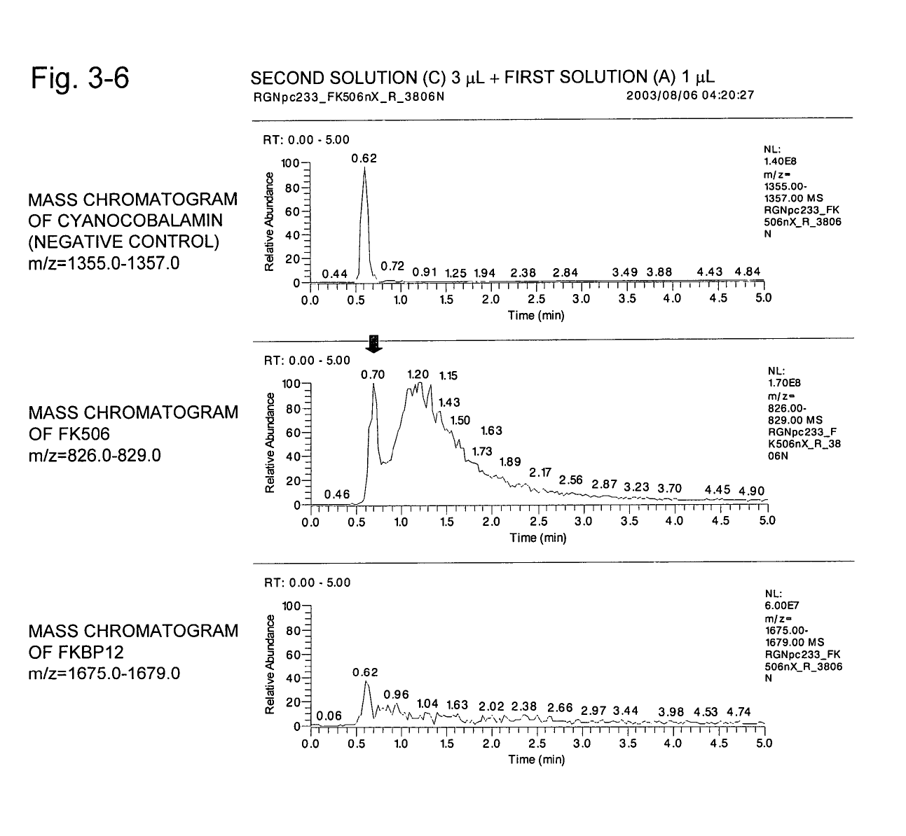

As seen from FIGS. 3-1 to 3-6, specific changes corresponding to interactions between the compounds FK506 and FKBP12 were observed in the mass chromatograms by introducing the Second Solution (c) and the First Solution (c) continuously. That is, when human FKBP12 is the first substance, peaks (peaks indicated by the arrows in FIGS. 3-4 to 3-6) appeared in the mass chromatograms of FK506. This shows that when FKBP12 passed over FK506 in the column, a portion of FK506 was bound to FKBP12 and was eluted with the protein from the column, which means there exists an interaction between FK506 and FKBP12.

As seen from FIGS. 4-1 to 4-3, specific changes corresponding to interactions between the compounds J-8 and bovine Calmodulin were also observed in the mass chromatograms by introducing the Second Solution (b) and the First Solution (b) continuously. That is, when bovine calmodulin is the first substance, rising baselines (rising baselines indicated by the arrows in FIGS. 4-1 to 4-3) appeared earlier. This shows that when calmodulin passed over J-8 in the column, a portion of J-8 interacted with calmodulin and was eluted earlier from the column, which means there exists an interaction between J-8 and calmodulin.

Also as shown in FIGS. 3-1 to 3-6 and FIGS. 4-1 to 4-3, the increased amounts of the injected second substance such as 1 μL, 2 μL and 3 μL increased the intensities of the mass chromatograms for the compounds, so that interaction analyses appeared more clearly with the increased amounts of the injected second substance.

Measurement 2

Interaction Analysis with Combined Second Substances

(Sample Preparation)

As first solutions with first substances, solutions containing a protein were prepared with compositions as follows respectively.

First Solution (a);
No first substance
500 μM ADA buffer solution (pH 6.5)

First Solution (b);
first substance: Bovine Calmodulin
50 μM Bovine Calmodulin
500 μM ADA buffer solution (pH 6.5)
100 μM CaCl₂

First Solution (c);
first substance: Human Calmodulin
50 μM Human Calmodulin
500 μM ADA buffer solution (pH 6.5)
100 μM CaCl₂
   As second solutions with a plurality of second substance (second substances), solutions containing low molecular weight compounds were prepared with compositions as follows respectively.

Second Solution (a);
No second substances
5% DMSO

38 kinds of second solutions (b); (each solution contains second substances which were combined with 5 to 8 kinds of compounds)
with second substances
25 μM each of 5 to 8 kinds of compounds
5% DMSO
   As the second solutions (b), 38 kinds of second solutions (Multi02-001 to Multi02-038) containing 5 to 8 kinds of compounds as second substances were used. For example, the second solution Multi02-001 contains 8 compounds (second substances) each of which is coded with Multi02-001A, 001B, 001C, 001D, 001E, 001F, 001G and 001H in an amount of 25 μM respectively, which compose the second substances. In the same way, 290 kinds of compounds (second substances) were combined and 38 kinds of second solutions Multi02-001 to Multi02-038 were prepared.
   (Measurement and Result)

The second solution and the first solution were introduced continuously into the TSK super SW2000 column in an amount of 1 μL respectively by the apparatus for analyzing interactions and the Mixing-in-Column method to obtain measurements for mass chromatograms of each compound (first substances and second substances). The results are shown in FIGS. 5-1 to 5-4 which showed differences between the chromatograms. As seen in FIGS. 5-1 to 5-4, with respect to the four compounds, Multi02-022E, Multi02-023G, Multi02-026C and Multi02-038G among the compounds in the second substances, specific changes were observed in the mass chromatograms when one of bovine calmodulin (b) or human calmodulin (c) was the first substance, as compared to the control (a) without calmodulin. That is, the rising baselines (the points indicated by the broken lines in FIGS. 5-1 to 5-5) in the chromatograms of the four compounds appeared earlier when calmodulin is the first substance. This means that when calmodulin passed over the combined second substances in the column, the four compounds interacted with calmodulin and were eluted from the column earlier.

### Example 2

Apparatus for Analyzing Interactions

In Example 2, the apparatus in Example 1 was modified partially to have a configuration to introduce a plurality of first solutions.

(1) Configuration of Auto Injector (HTC-PAL)

The auto injector was an HTC-PAL (CTC Analytics AG) with the same configuration as that of Example 1 except including a sample loop of 10 μL.

In this Example, Mixing-in-Column method in a sequence below was programmed with an editing macro of an autosampler HTC-PAL (CTC Analytics AG). Mixing-in-Column method:

1) Clean syringe inside (solvent 1:50% methanol-water solution);

2) Clean syringe inside (solvent 2: MiliQ water);

3) Aspirate 1 μL of a second substance(s) solution from a well specified by sample sequence;

4) Aspirate 0.2 μL of air;

5) Clean syringe outside (solvent 1:50% methanol-water solution);

6) Clean syringe outside (solvent 2: MiliQ water);

7) Aspirate 1 μL of a first substance(s) solution from a vial specified by sample sequence;

8) Repeat sequence from 4) to 7) required (n) times;

9) Inject the 1.0+(1.2×n) μL into injection port;

10) Clean syringe inside (solvent 1:50% methanol-water solution);

11) Clean syringe inside (solvent 2: MiliQ water);

12) Clean injection port (solvent 1:50% methanol-water solution); and

13) Clean injection port (solvent 2: MiliQ water),

where the amounts of first solution, second solution, and air are changeable.

An example of sequence controls in the autosampler HTC-PAL is shown below, where three kinds of first solutions containing three kinds of proteins No. 3, No. 4, and No. 5 are aspirated continuously.

syringe: 10 ul

LC-Asp_Chems_in 384Col(2,0.2)

LC-Asp_Separate_ProteinA_in 54Vials(0.2,CStk 1-01,3,1)

LC-Asp_Separate_ProteinA_in 54Vials(0.2,CStk 1-01,4,1)

LC-Inj_Separate_Protein_in 54Vials(2,2,0.2,CStk 1-01,5,1,4.6)

[MACRO LC-Asp_Chems_in 384Col] Pre Clean with Solvent 0;2;0;99

Post Air Volume (μl); 1;0;SYR.Max Volume

WAIT_SYNC_SIG(Start,)

CLEAN_SYR(Wash1,Pre Clean with Solvent,,,,,,,)

CLEAN_SYR(Wash2,Pre Clean with Solvent,,,,,,,)

GET_SAMPLE(SL.tray,SL.index,SL.volume,Post Air Volume,,, 2,,2,,Off,,,)

MOVETO_OBJECT(Wash1,,,)

MOVETO_OBJECT(Wash2,,,)

MOVETO_OBJECT(Wash1,,,)

MOVETO_OBJECT(Wash2,,,)

[MACRO LC-Asp_Separate_ProteinA_in 54Vials]

Post Air Volume (μl);1;0;SYR.Max Volume

Protein Rack Position;TRAY

ProteinA Index ( ); 1; 1;54

Protein Volume (μl);1;0;SYR.Max Volume

GET_SAMPLE(Protein Rack Position,ProteinA Index,Protein Volume,Post Air Volume,,,2,,2,,Off,,,)

MOVETO_OBJECT(Wash1,,,)

MOVETO_OBJECT(Wash2,,,)

MOVETO_OBJECT(Wash1,,,)

MOVETO_OBJECT(Wash2,,,) [MACRO LC-Inj_Separate_Protein_in 54Vials]

Valve Clean with Solvent ( );2;0;99

Post Clean with Solvent ( );2;0;99

Post Air Volume (μl); 1;0;SYR.Max Volume

Protein Rack Position;TRAY

Protein Index ( );1;1;54

Protein Volume (μl);1;0;SYR.Max Volume

Injection Volume (μl);5;0;SYR.Max Volume

GET_SAMPLE(Protein Rack Position,Protein Index,Protein Volume, 0,,,2,,2,,Off,,,)

MOVETO_OBJECT(LC Vly1,,,)

WAIT_FOR_DS( )

INJ_SAMPLE(LC Vlv1,Inject,Injected,,Injection Volume, 500,,, 1,)

CLEAN_SYR(Wash1,Post Clean with Solvent,,,,,,,)

CLEAN_INJ(Wash1,LC Vly1,Valve Clean with Solvent,,,,,,,,,)

CLEAN_SYR(Wash2,Post Clean with Solvent,,,,,,,)

CLEAN_INJ(Wash2,LC Vly1,Valve Clean with Solvent,,,,,,,,,)

[MACRO METHOD ENTRY]

LOCK_TERMINAL(On,)

CLEANUP(Wash1,Off, Off,On, Off,On, Off, Off,)

[MACRO METHOD EXIT]

CLEANUP(Wash1,Off,Off,Off,On,Off,Off,On,)

LOCK_TERMINAL(Off,)

Measurement 3

Interaction Analysis for proteins with Mixing-in-Column method in Example 2

(Sample Preparation)

First solutions containing a protein was prepared with a composition as follows respectively. Hereinafter, a substance in the first solution is referenced to as a first substance.

First solutions containing a protein as a first substance were prepared with compositions as follows respectively.

First Solution (a);
No first substance (Reference)
10 mM of ADA buffer solution (pH 6.5)
100 μM CaCl₂

First Solution (b);
first substance: Bovine Calmodulin
100 μM Bovine Brain Calmodulin (CaM; manufactured by Calbiochem)
100 μM CaCl₂

First Solution (c);
first substance: Human FKBP12
100 μM Human FKBP12
10 mM ADA buffer solution (pH 6.5)

First Solution (d);
first substance: Human Serum Albumin
100 μM Human Serum Albumin (HSA; manufactured by Sigma Chemicals Co.)
10 mM ADA buffer solution (pH 6.5)
   Second solutions containing a low molecular weight compound as a second substance were prepared with compositions as follows respectively.

Second Solution (a);
No second substance (Reference)
5% DMSO
50 μM Cyanocobalamin (Reference)

Second Solution (b);
second substance: J-8
100 μM J-8
50 μM Cyanocobalamin (Reference)
5% DMSO

Second Solution (c);
second substance: FK506
50 μM FK506
50 μM Cyanocobalamin (Reference)
5% DMSO

Second Solution (d);
second substance: Ascomycin
50 μM Ascomycin
50 μM Cyanocobalamin (Reference)
5% DMSO
   (Measurement and Result)

A second solution and a first solution(s) consisting of a plurality of solution samples were introduced into the TSK super SW2000 column by the apparatus illustrated in Example 2 and the Mixing-in-Column method to obtain mass chromatograms of each compound (first substances and second substances). In this Example, solutions were introduced in the orders as follows, where between the solutions was interposed a gaseous spatial sample (air).

Second Solution (c)→First Solution (a)

Second Solution (c)→First Solution (d)→First Solution (d)

Second Solution (c)→First Solution (c)

Second Solution (c)→First Solution (c)→First Solution (d)

Second Solution (c)→First Solution (d)→First Solution (c)

The results are shown in FIGS. 6-1 to 6-5. As shown in FIGS. 6-1 to 6-5, when FK506 (second substance)-air-HSA-air-HSA are continuously introduced into the column, there is no change in the result compared to when FK506 (second substance)-air-ADA buffer solution are continuously introduced. However, when FK506-air-HSA-air-FKBP12 are continuously introduced into the column, and when FK506-air-FKBP12-air-HSA are continuously introduced into the column, peaks (the peaks indicated by the arrows in FIGS. 6-3 to 6-5) appeared in the mass chromatograms of FK506 in the same way as when FK506-air-FKBP12 were continuously introduced. This means that, among the introduced plurality of solution samples, when a solution sample with FKBP12 passed over FK506 in the column, a portion of FK506 bound to FKBP12 and was eluted earlier with the protein from the column. Therefore this example shows that even if first solutions consisting of a plurality of solution samples are introduced after a second solution continuously, when at least one of the plurality of solution samples includes a substance which interacts with the second substance, the interaction is able to be detected.

Similarly, each solution was introduced in the order as follows, where between the solutions was interposed a gaseous spatial sample (air).

Second Solution (b)→First Solution (a)

Second Solution (b)→First Solution (d)→First Solution (d)

Second Solution (b)→First Solution (b)

Second Solution (b)→First Solution (b)→First Solution (d)

Second Solution (b)→First Solution (d)→First Solution (b)

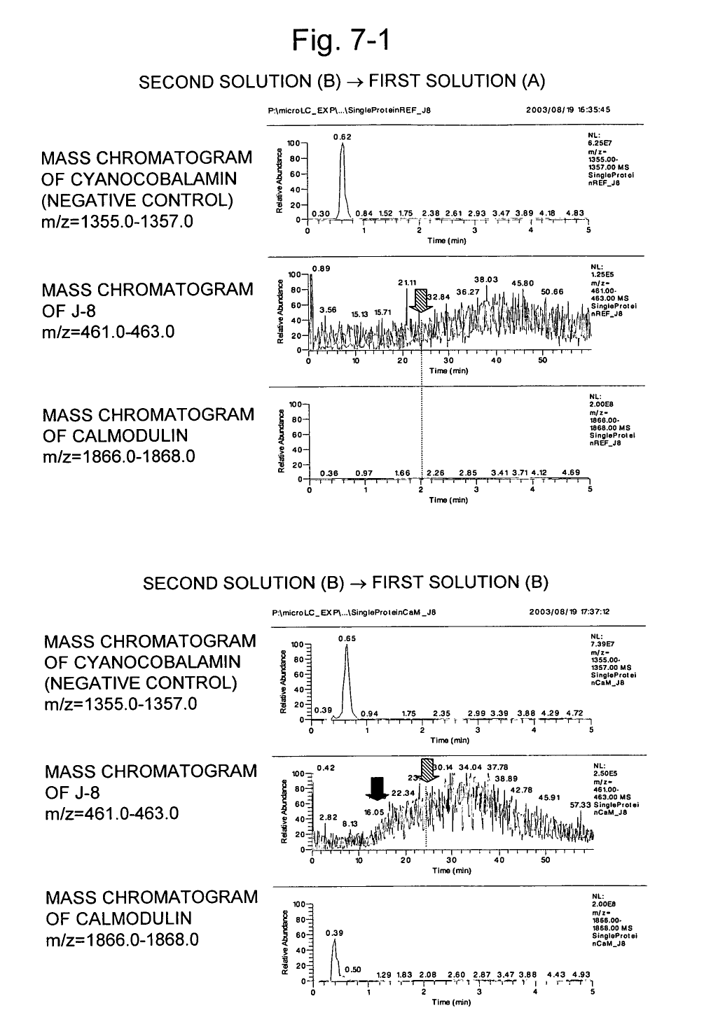

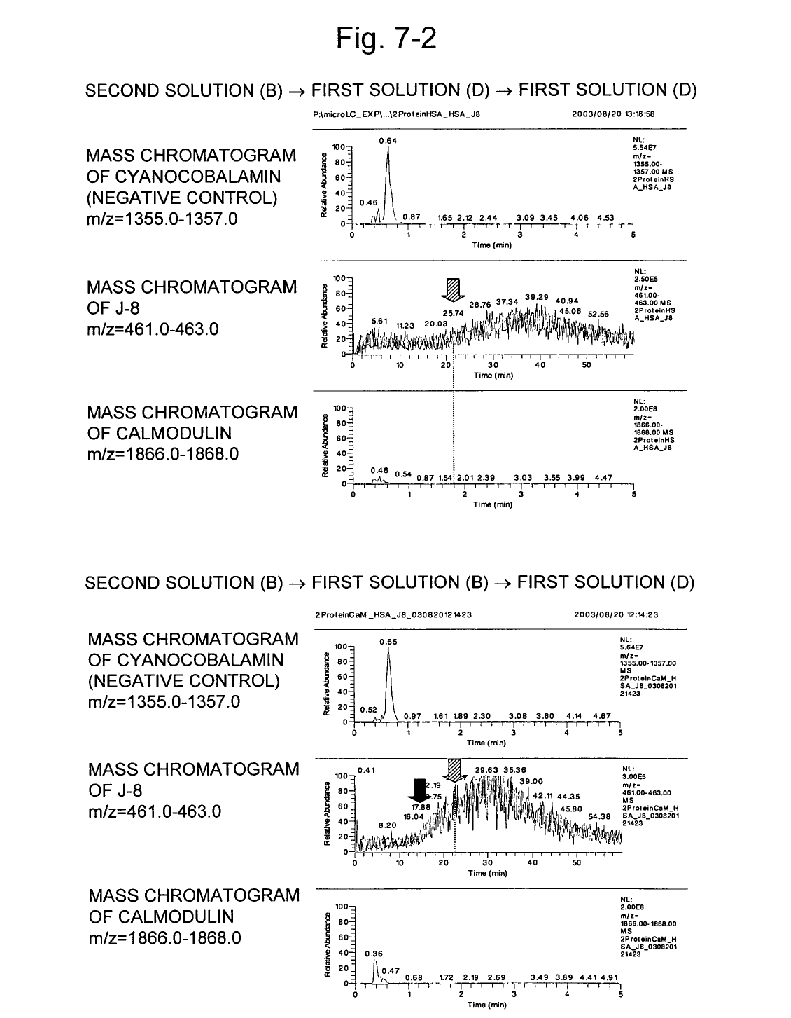

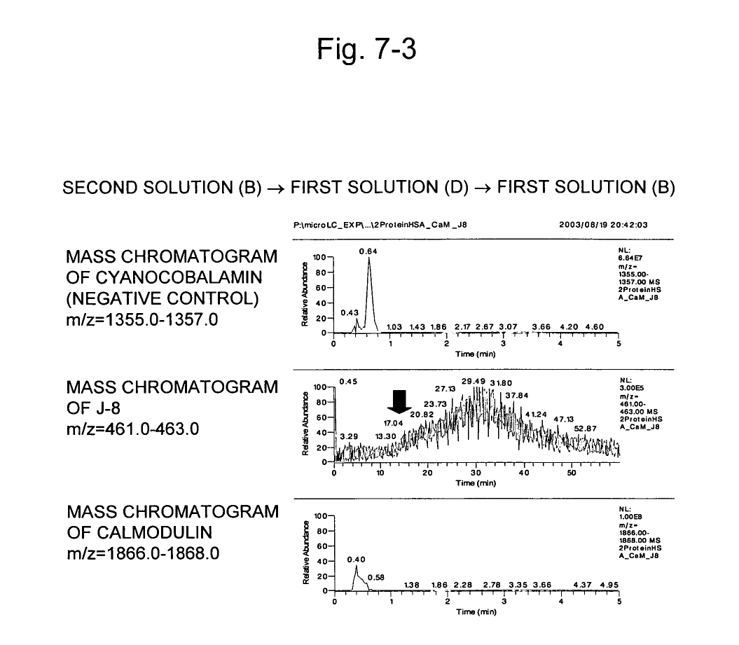

The results are shown in FIGS. 7-1 to 7-3. As shown in FIGS. 7-1 to 7-3, when J-8 (second substance)-air-HSA-air-HSA are continuously introduced into the column, there is no change in the mass chromatogram compared to when J-8-air-ADA buffer solution are continuously introduced. However, when J-8-air-HSA-air-CaM are continuously introduced into the column, and when J-8-air-CaM-air-HSA are continuously introduced into the column, rising baselines (the points indicated by the arrows in FIGS. 7-1 to 7-3) appeared earlier in the mass chromatograms of J-8 in the same way as when J-8-air-CaM were continuously introduced. This means that, among the introduced plurality of solution samples, when a solution sample with calmodulin passed over J-8 in the column, a portion of J-8 interacted with calmodulin and was eluted earlier from the column. Therefore this example also shows that even if first solutions consisting of a plurality of solution samples are introduced after a second solution continuously, when at least one of the plurality of solution samples includes a substance which interacts with the second substance, the interaction is able to be detected.

Next, another case was examined where a second solution and first solutions consisting of three kinds of solution samples are introduced continuously. Each solution was introduced in the order as follows, where between the solutions was interposed a gaseous spatial sample (air).

Second Solution (c)→First Solution (a)→First Solution (a)→First Solution (a)

Second Solution (c)→First Solution (d)→First Solution (d)→First Solution (d)

Second Solution (c)→First Solution (c)→First Solution (d)→First Solution (d)

Second Solution (c)→First Solution (d)→First Solution (c)→First Solution (d)

Second Solution (c)→First Solution (d)→First Solution (d)→First Solution (c)

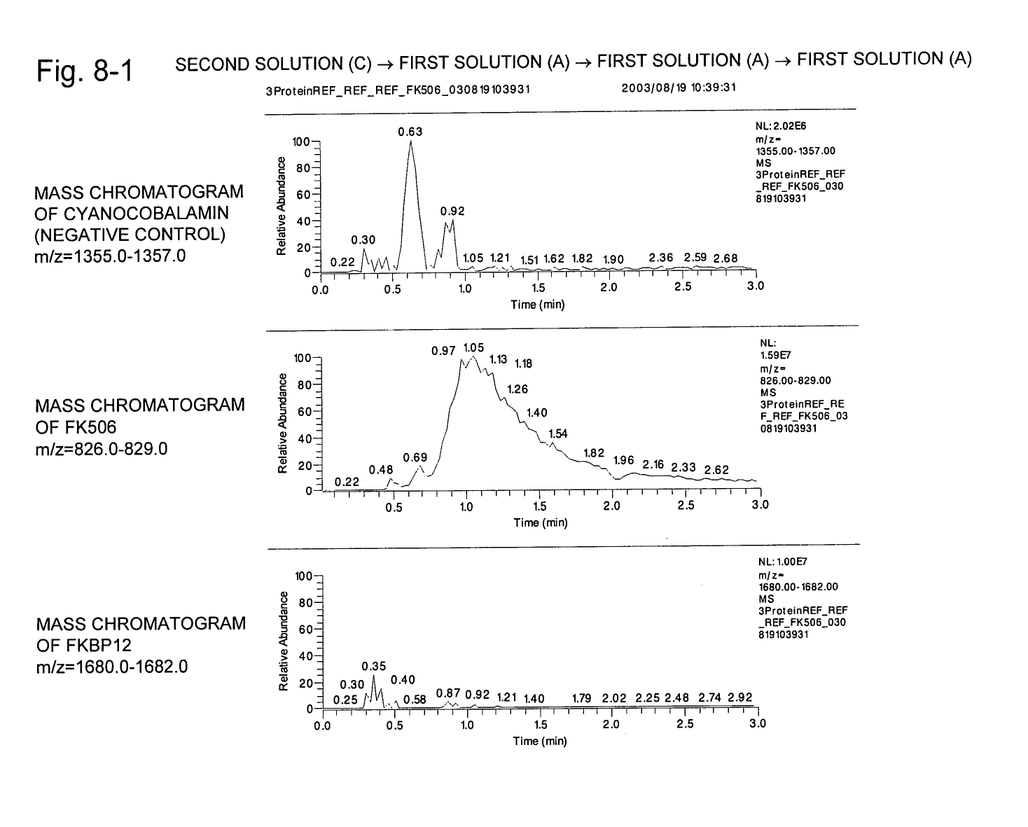

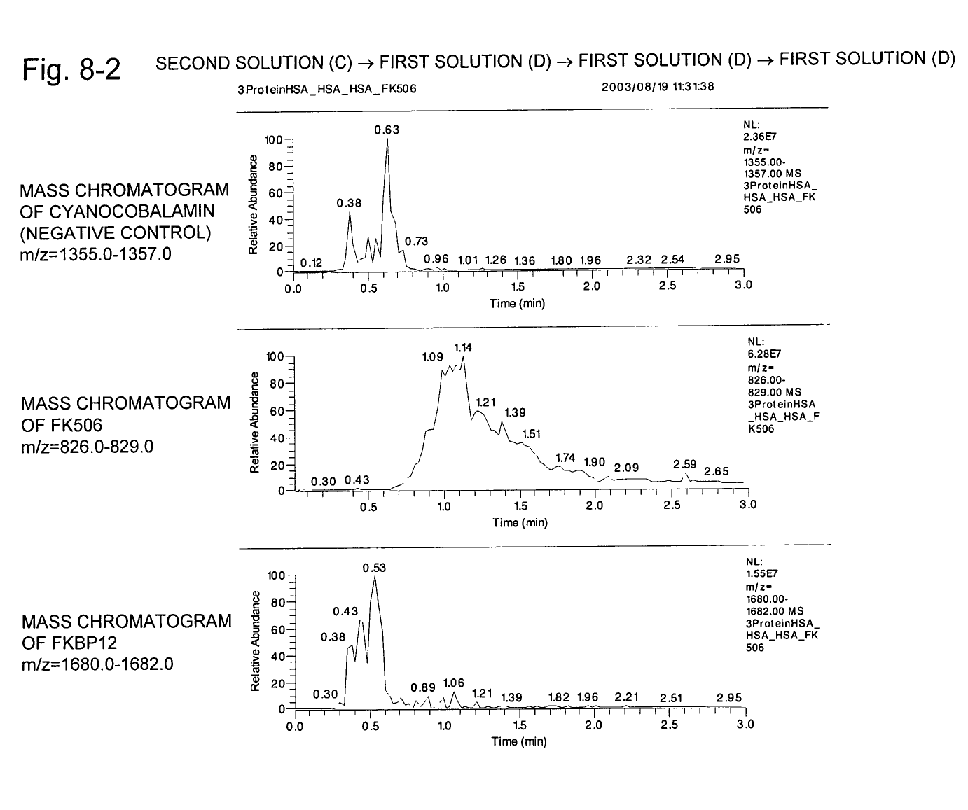

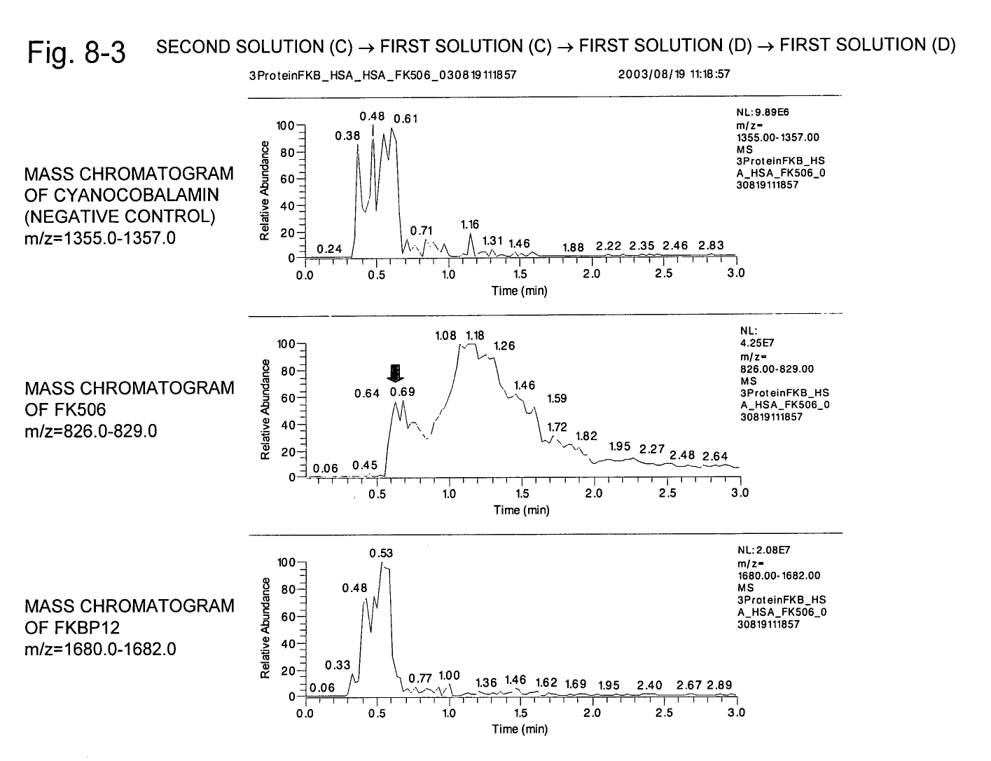

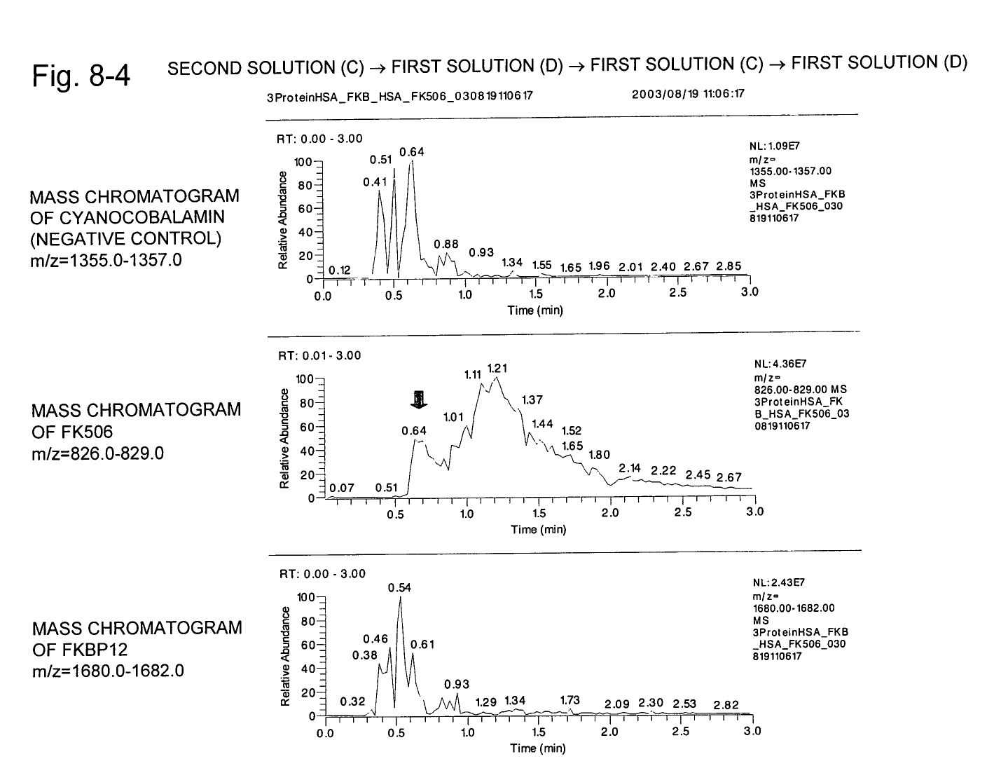

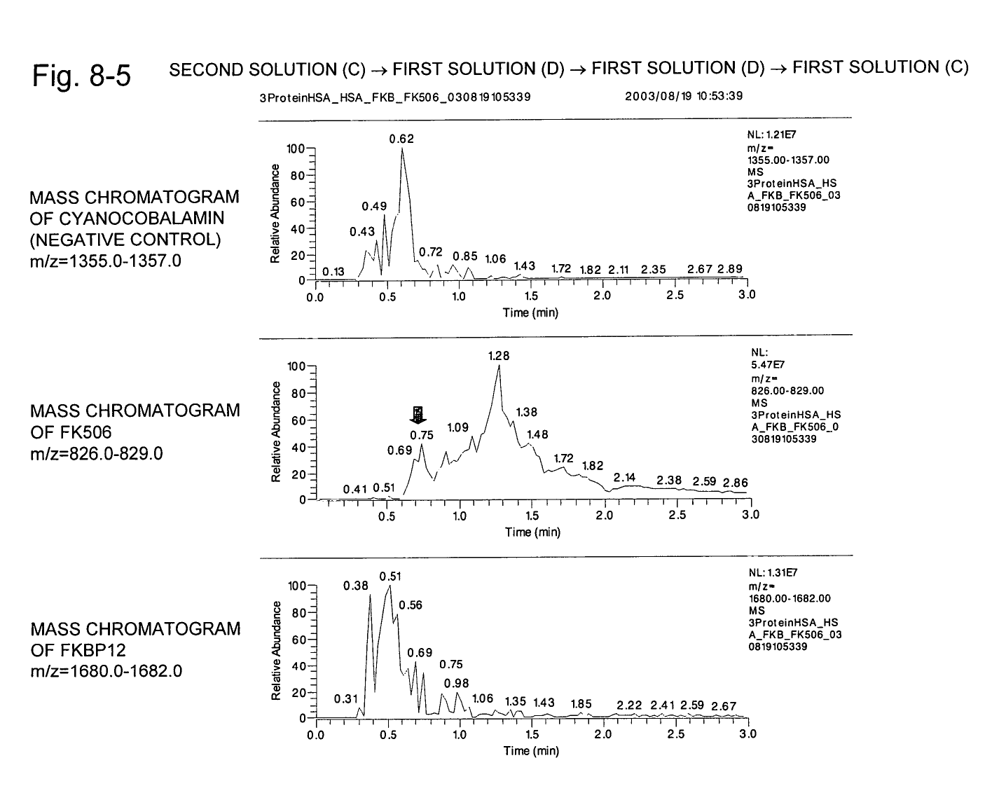

The results are shown in FIGS. 8-1 to 8-5. As shown in FIGS. 8-1 to 8-5, when FK506 (second substance)-air-HSA-air-HSA-air-HSA are continuously introduced into the column, there is no change in the mass chromatogram compared to when FK506-air-ADA buffer solution-air-ADA buffer solution-air ADA buffer solution are continuously introduced. However, when one of the three solution samples includes a substance which interacts with FK506 (second substance), peaks (the peaks indicated by the arrows in FIGS. 8-1 to 8-3) appeared in the mass chromatograms of FK506. Therefore this result also shows that even if first solutions consisting of a plurality of solution samples are introduced after a second solution continuously, when at least one of the plurality of solution samples includes a substance which interacts with the second solution, the interaction is able to be detected.

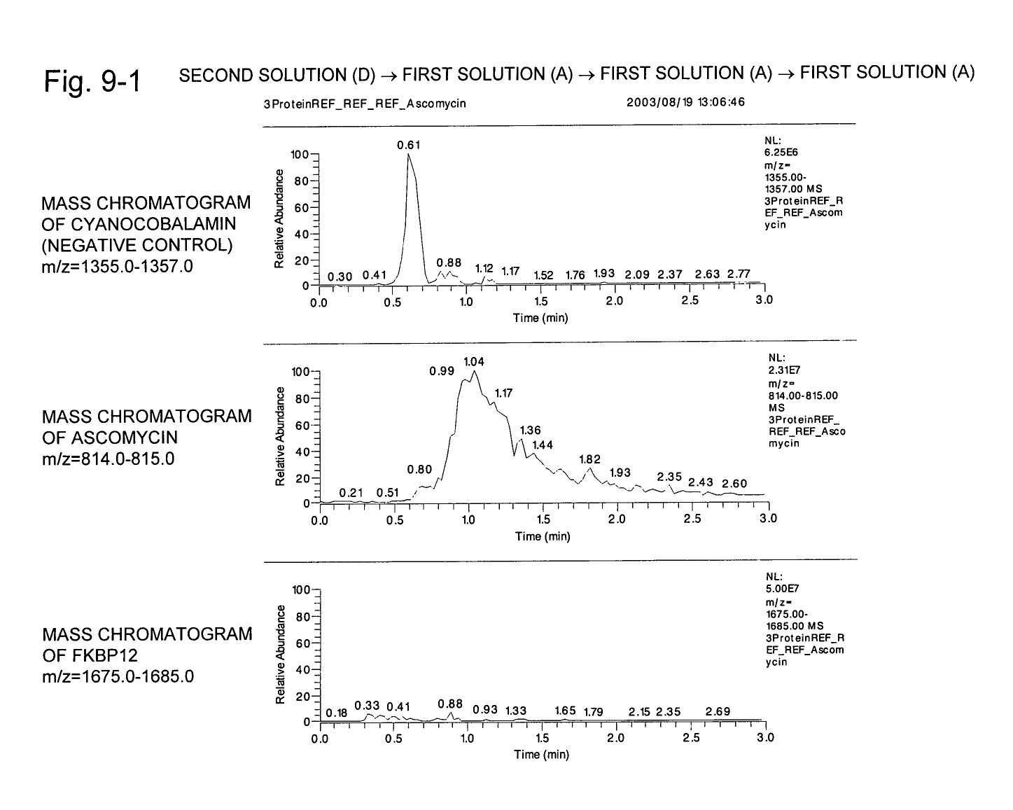

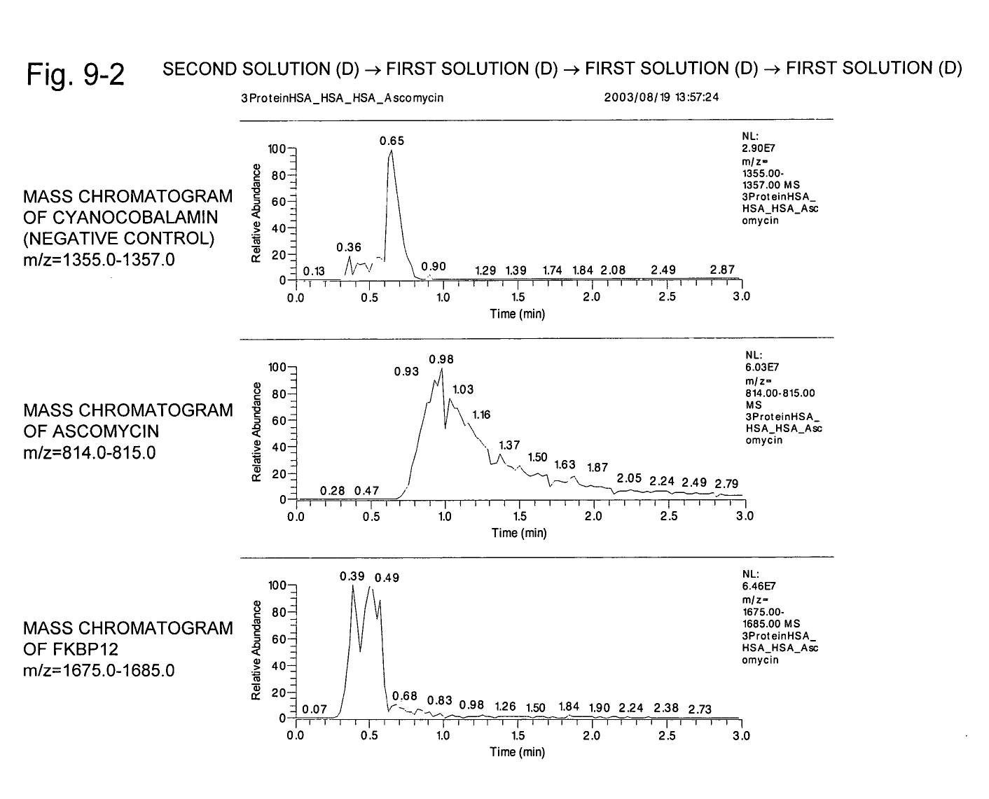

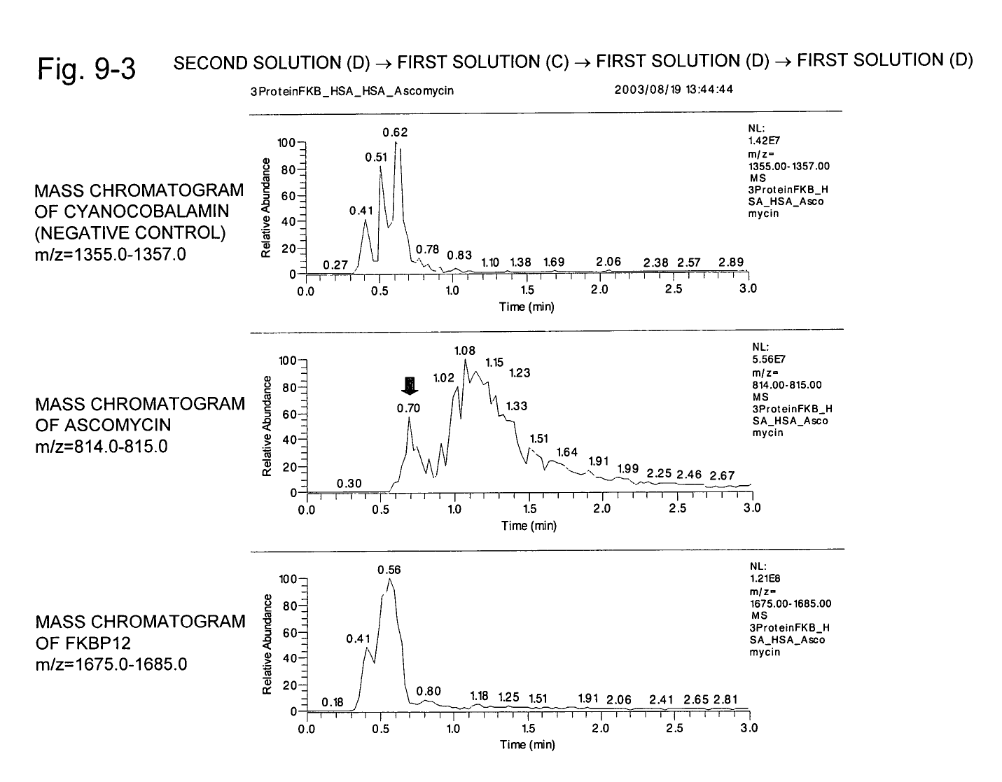

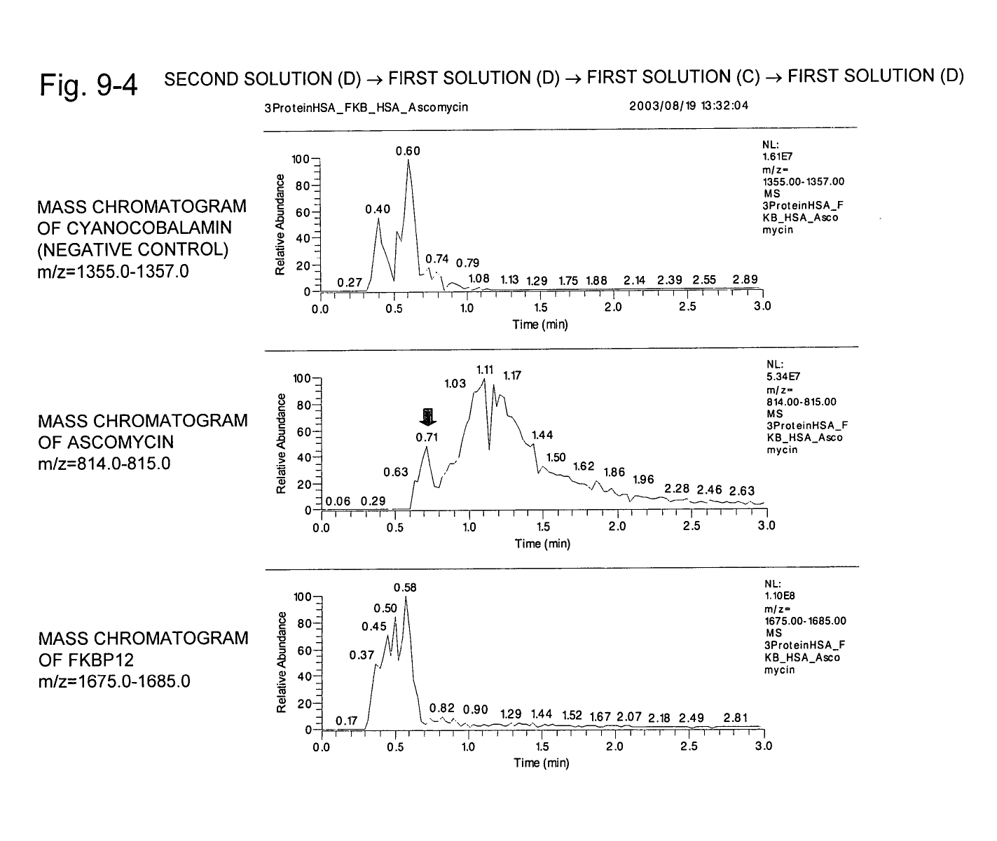

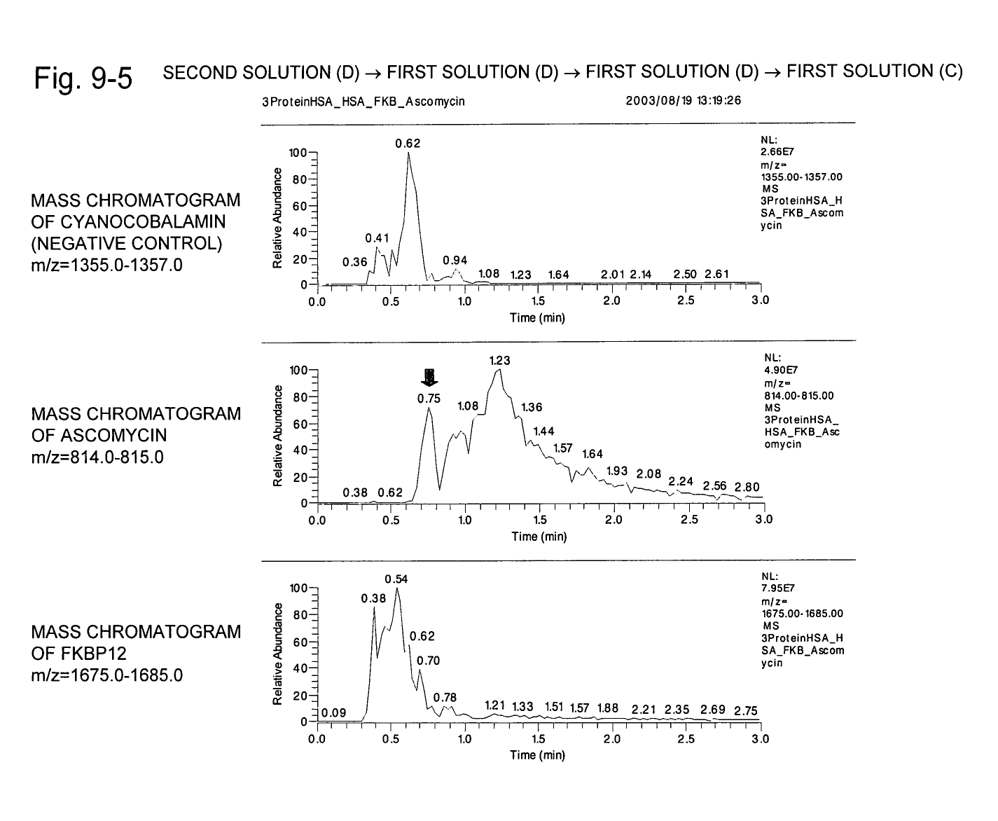

Another result of a case is shown in FIGS. 9-1 to 9-5 where Ascomycin was used as a second substance instead of FK506. As shown in FIGS. 9-1 to 9-5, this result also shows that the interaction between Ascomycin and FKBP12 is able to be detected.

### Example 3

Apparatus for Analyzing Interactions

In Example 3, as shown in FIG. 2-2, an apparatus for analyzing interactions is configured to have a 2^{nd} stage of a separation channel (column for interaction analysis) mounted downstream of the 1^{st} stage of the separation channel (column for separation), where after a first solution is introduced from a first injector into the 1^{st} stage of the separation channel 1, the fraction eluted from the 1^{st} stage of the separation channel 1 is introduced into the 2^{nd} stage of a separation channel 1 with a second solution which is introduced from a second injector, so that a chromatogram is detected with respect to a substance contained in the second solution eluted from the 2^{nd} stage of a separation channel 1 to determine with which fraction eluted from the 1^{st} stage the substance in the second solution interacted. Specifically, the apparatus for analyzing interactions in this example includes a column TSK super SW3000 (column size: 1.0 ID×100 mm, manufactured by Tosoh Corporation) for size exclusion chromatography as the 1^{st} stage of a separation channel 1, and a column TSK super SW2000 for size exclusion chromatography (column size: 1.0 ID×30 mm, manufactured by Tosoh Corporation) as the 2^{nd} stage of the separation channel 1. The apparatus for analyzing interactions in this example also includes an auto injector Waters 2777 Sample Manager (manufactured by CTC Analytics AG) as a container section 3, and an LC pump Agilent 1100 (manufactured by Yokogawa Analytical Systems Inc.) and an Micro 21LC (manufactured by Japan Spectroscopy Co.) as an introduction device 4. The apparatus for analyzing interactions in this example further includes an ion trap mass spectrometer LCQ deca XP (manufactured by Thermo Electron Co.) as a detection device 5.

(1) Configuration of Auto Injector (Waters 2777)

The auto injector Waters 2777 (CTC Analytics AG) includes a first injector with a sample loop of 40 μL being connected thereto, a second injector with a sample loop of 10 μL being connected thereto, a syringe of 10 μL, and sample trays with a cooling unit. A first solution was introduced by 50 μL into a 2 mL screw vial with a conical insert of 100 μL inserted therein. A screw cap with a septum was put on the vial and the vial was placed in a 54 vial rack and the rack was set in one of the sample trays. A second solution was introduced by 40 μL into each well of a 384 well-microplate, and the microplate was covered with an aluminum seal and set in another sample tray. The sample trays were set at the temperature of 10 degrees C.

A analysis method (hereinafter referenced to as Two-Stage Mixing-in-Column Method) in a sequence below was programmed with a macro editor of an auto injector Waters 2777 (CTC Analytics AG). The Two-Stage Mixing-in-Column Method consists of two methods: a method “Inj 1” for injecting a first solution from a first injector; and a method “Inj 2” for injecting a second solution from a second injector repeatedly at predetermined intervals. In the sample sequence by the auto injector Waters 2777, a first solution held in a container section (sample tray) was introduced from the first injector into the 1^{st} stage of the separation channel 1 with the method “Inj 1”, and after that, a second solution held in the container section (sample tray) was introduced from the second injector into the 2^{nd} stage of the separation channel with the method “Inj 2”.

An Example of Sample Sequence in Two-Stage Mixing-in-Column Method

| | | | | Tray | |
|---|---|---|---|---|---|
| # | Method | Volume | Injector | Position | Vial Position |
| 1. | Inj 1 | 1 μL | LC Vlv1 | CStk1-01 | 1 where first solution |
| | | | | | is held |
| 2. | Inj 2 | 1 μL | LC Vlv2 | CStk1-03 | 1 where second |
| | | | | | solution is held |

When there are a large number of samples, a sample sequence was programmed to inject a first solution and then a second solution in the same way with the above by repeating the Inj 1 and the Inj 2.

Two-Stage Mixing-in-Column Method
<Method Inj 1> (a method to inject a first solution once)
syringe:10μl
LC-Inj(1,1,0,8,4,1,SL.injector,8,500,500,1,0,1,1)
Clean Syringe(Wash1,2)
Clean Syringe(Wash2,2)
[MACRO LC-Inj]
Pre Clean with Solvent 1 ( );0;0;99
Pre Clean with Solvent 2 ( );0;0;99
Pre Clean with Sample ( );0;0;99
Eject Speed (μl/s);SYR.Eject Speed;SYR.Min Speed;SYR.Max Speed
Filling Speed (μl/s);Syr.Fill Speed;Syr.Min Speed;Syr.Max Speed
Filling Strokes ( );1;0;99
Inject to;INJECTOR;
Injection Speed (μl/s);SYR.Inject Speed;SYR.Min Speed;SYR.Max Speed
Pre Inject Delay (ms);500;0;99000
Post Inject Delay (ms);500,0,99000
Post Clean with Solvent 1 ( );1;0;99
Post Clean with Solvent 2 ( );1;0;99
Valve Clean with Solvent 1 ( );1;0;99
Valve Clean with Solvent 2 ( );1;0;99
WAIT_SYNC_SIG(Start,)
CLEANUP(Wash1,Off,Off,On,Off,Off,Off,Off,)
CLEAN_SYR(Wash1,Pre Clean with Solvent 1,,,,,Eject Speed,,)
CLEAN_SYR(Wash2,Pre Clean with Solvent 2,,,,,Eject Speed,,)
REPEAT(Pre Clean with Sample,)
GET_SAMPLE(SL.tray,SL.index,SYR.Max Volume*0.2,,,,Filling Speed,,,0,Off,,,)
CLEANUP(Wash1,Off,Off,Off,Off,Off,On,Off,)
PUT_SAMPLE(Waste,1,,,Eject Speed,,)
END( )
GET_SAMPLE(SL.tray,SL.index,SL.volume,,,SYR.Fill Volume,Filling Speed, 2000,, Filling
Strokes, Off,,,)
CLEANUP(Wash1,Off,Off,Off,Off,Off,On,Off,)
INJ_SAMPLE(Inject to,Inject,Injected,,,Pre Inject Delay,Injection Speed,Post Inject Delay,5,)
SEND_REM_SYNC( )
START_TIMER(5,)
CLEAN_SYR(Wash1,Post Clean with Solvent 1,,,,,Eject Speed,,)
CLEAN_SYR(Wash2,Post Clean with Solvent 2,,,,,Eject Speed,,)
CLEAN_INJ(Wash1,Inject to,Valve Clean with Solvent 1,,,,,,,,Injection Speed,)
CLEAN_INJ(Wash2,Inject to,Valve Clean with Solvent 2,,,,,,,,Injection Speed,)
MOVETO_OBJECT(Home,,,)
[MACRO Clean Syringe]
Clean Wash Station;WASH_STATION;
Number of Clean Cycles ( );1;0;99
CLEAN_SYR(Clean Wash Station,Number of Clean Cycles,,,,,,,)
[MACRO METHOD ENTRY]
CLEANUP(Wash1,Off,Off,Off,Off,Off,Off,Off,)
[MACRO METHOD EXIT]
CLEANUP(Wash1,Off,Off,Off,On,Off,Off,On,)
<Method Inj2> (a method to inject a second solution repeatedly at predetermined intervals)
syringe:10ul
LC-Inj_Repeat_NoSync(1,1,20,5,1,LC Vlv1,10,1,1,1,1,9,120)
Clean Syringe(Wash1,2)
Clean Syringe(Wash2,2)
[MACRO LC-Inj_Repeat_NoSync]
Pre Clean with Solvent 1 ( );0;0;99
Pre Clean with Solvent 2 ( );0;0;99
Eject Speed (μl/s);SYR.Eject Speed;SYR.Min Speed;SYR.Max Speed
Filling Speed (μl/s);Syr.Fill Speed;Syr.Min Speed;Syr.Max Speed
Filling Strokes ( );1;0;99
Inject to;INJECTOR;
Injection Speed (μl/s);SYR.Inject Speed;SYR.Min Speed;SYR.Max Speed
Post Clean with Solvent 1 ( );1;0;99
Post Clean with Solvent 2 ( );1;0;99
Valve Clean with Solvent 1 ( ),1;0;99
Valve Clean with Solvent 2 ( );1;0;99
Injection Repeat ( );1;0;999
Injection Interval (s);120;0;9999
WAIT_SYNC_SIG(None,)
CLEANUP(Wash1,Off,Off,On,Off,Off,Off,Off,)
CLEAN_SYR(Wash1,Pre Clean with Solvent 1,,,,,Eject Speed,,)
CLEAN_SYR(Wash2,Pre Clean with Solvent 2,,,,,Eject Speed,,)
REPEAT(Injection Repeat,)
GET_SAMPLE(SL.tray,SL.index,SL.volume,,,SYR.Fill Volume,Filling Speed, 2000,, Filling
Strokes,Off,,,)
CLEANUP(Wash1,Off,Off,Off,Off,Off,On,Off,)
MOVETO_OBJECT(Inject to,1,,)
SWITCH_INJ(Inject to,Active,,)
PUT_SAMPLE(Inject to, 1,,SL.volume,,,)
SWITCH_INJ(Inject to,Standby,1,)
SEND_REM_SYNC( )
CLEAN_SYR(Wash1,Post Clean with Solvent 1,,,,,Eject Speed,,)
CLEAN_SYR(Wash2,Post Clean with Solvent 2,,,,,Eject Speed,,)
WAIT_TIMER(1,Injection Interval,)
END( )
CLEAN_INJ(Wash1,Inject to,Valve Clean with Solvent 1,,,,,,,,Injection Speed,)
CLEAN_INJ(Wash2,Inject to,Valve Clean with Solvent 2,,,,,,,,Injection Speed,)
MOVETO_OBJECT(Home,,,)
[MACRO Clean Syringe]
Clean Wash Station;WASH_STATION;
Number of Clean Cycles ( );1;0;99
CLEAN_SYR(Clean Wash Station,Number of Clean Cycles,,,,,,,)
[MACRO METHOD ENTRY]
CLEANUP(Wash1,Off,Off,Off,Off,Off,Off,Off,)
[MACRO METHOD EXIT]
CLEANUP(Wash1,Off,Off,Off,On,Off,Off,On,)

(2) Configurations of LC pump (Agilent 1100) and LC pump (micro 21LC), and Ion Trap Mass Spectrometer LCQ deca X

The auto injector Waters 2777 has a first injector and a second injector. A solution feed line for column equilibrating from a binary pump (B pump) in an LC pump (Agilent 1100) was connected to the inlet port of the first injector. To the outlet port of the first injector was connected to the 1^{st} stage column TSK super SW3000 (ID1.0×100 mm) for size exclusion chromatography, and the downstream side of the column was connected to one of the inlet ports of a Nanotight Y Connector (Upchurch Scientific), the outlet port of which was connected to an upstream end of the 2^{nd} stage column TSK super SW2000 (ID1.0×30 mm) for size exclusion chromatography. While, a solution feed line for column equilibrating from a quaternary pump (Q pump) of an LC pump (Agilent 1100) was connected to an inlet ports of a second injector, and a line from an outlet port of the second injector was connected to the other inlet port of the Nanotight Y Connector (Upchurch Scientific). The downstream side of the 2^{nd} stage column TSK super SW2000 for size exclusion chromatography was connected to an ESI PROBE of an ion trap mass spectrometer LCQ deca XP via a Tee connector (PEEK Mixing Tee; GL Sciences Inc.). To the Tee connector was connected a solution feed line for ESI conditioning from a micro 21LC (Japan Spectroscopy Co.).

Then both of the binary pump (B pump) and the quaternary pump(Q pump) fed 10 mM ammonium acetate solution as a column equilibrating solution at 5 μL/min, and the micro 21LC pump fed 1.0% formic acid/methanol solution as an ESI conditioning solution at 2.5 μL/min.

(3) Measurement

The first solution and the second solution were automatically injected continuously from the vials and the sample wells with the above Two-Stage Mixing-in-Column Method to obtain measurements for mass chromatograms of the substances in the second solution. Thus, as an example, the first solution from the first injector was injected into the 1^{st} stage of the separation channel 1 (column for separation), and then the second solution from the second injector was injected into the 2^{nd} stage of the separation channel 1 (column for interaction analysis) repeatedly at timed intervals. As a result, the substance in the first solution was eluted from the 1^{st} stage of the separation channel 1 (column for separation) at predetermined elution time depending on to the substance characteristics, and was joined to the second solution which was injected repeatedly at timed intervals from the second injector 2, at the Nanotight Y Connector to be introduced into the 2^{nd} stage of the separation channel 1 (column for interaction analysis).

The substance(s) in the second solution injected repeatedly at timed intervals from the second injector 2 was eluted continuously from the 2^{nd} stage of the separation channel 1 (column for interaction analysis) and was constructed into a mass chromatogram in pulses with the ion trap mass spectrometer LCQ deca XP. If no substance which interacts with the substance(s) in the second solution is eluted from the 1^{st} stage of the separation channel 1 (column for separation), a similar mass chromatogram in pulses is detected at timed intervals. However, if a substance which interacts with the substance(s) in the second solution is eluted from the 1^{st} stage of the separation channel 1 (column for separation), there will be some change in the pulses of a mass chromatogram of the substance in the second solution at the 2^{nd} stage of the separation channel 1 (column for interaction analysis) where the interacting substance in the first solution passes over the substance in the second solution.

Measurement 4

Interaction Analysis with Two-Stage Mixing-in-Column Method

(Sample Preparation)

First solutions containing a protein as a first substance were prepared with compositions as follows respectively.

(a) No first substance (Reference)
10 mM ammonium acetate solution (pH 6.7)

(b) first substance: Human Serum Albumin
100 μM Human Serum Albumin (HSA; Sigma Chemicals Co.)
   Second solutions containing a low molecular weight compound as a second substance were prepared with a composition as follows respectively.

(a) No second substance (Reference)
5% DMSO

(b) second substance: Warfarin
100 μM Warfarin
5% DMSO
   (Measurement and Result)

A first substance was introduced from the first injector into the TSK super SW3000 column (column size: 10 ID×100 mm, Tosoh Corporation) by the apparatus illustrated in Example 3 and the Two-Stage Mixing-in-Column method, and after that a second substance was introduced from the second injector into a TSK super SW2000 (column size: 1.0 ID×30 mm, Tosoh Corporation) to obtain mass chromatograms of Warfarin compounds. The results are shown in FIGS. 10-1 to 10-6.

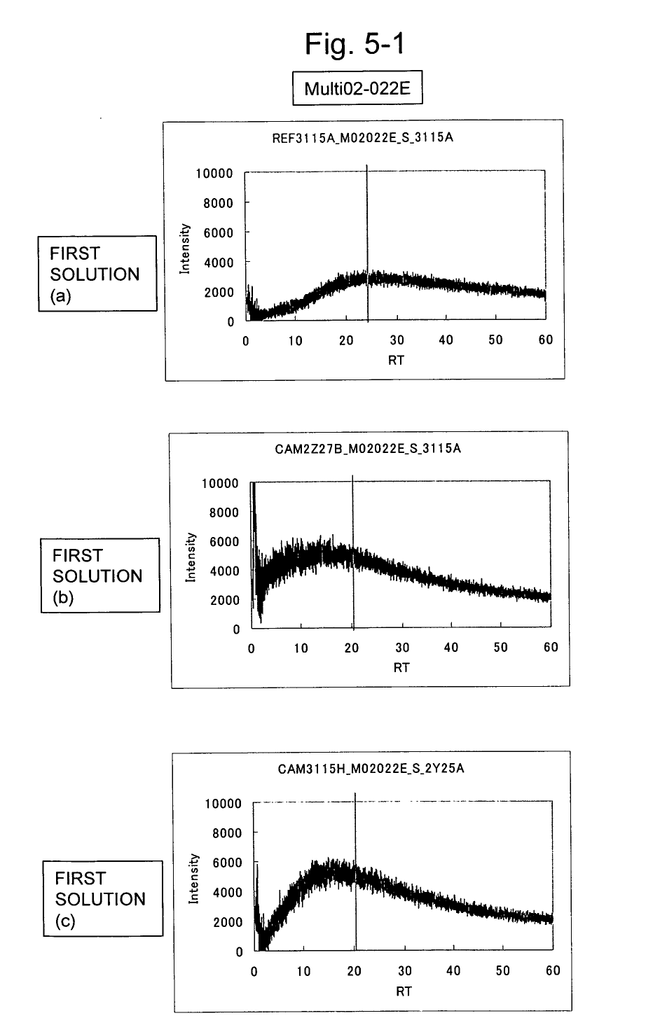

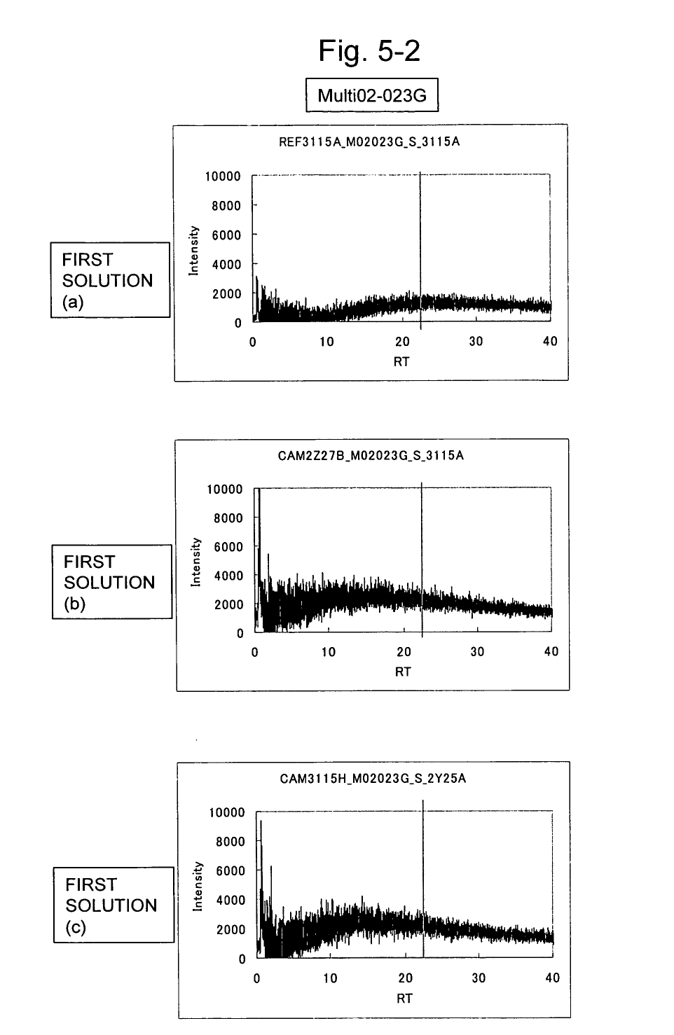

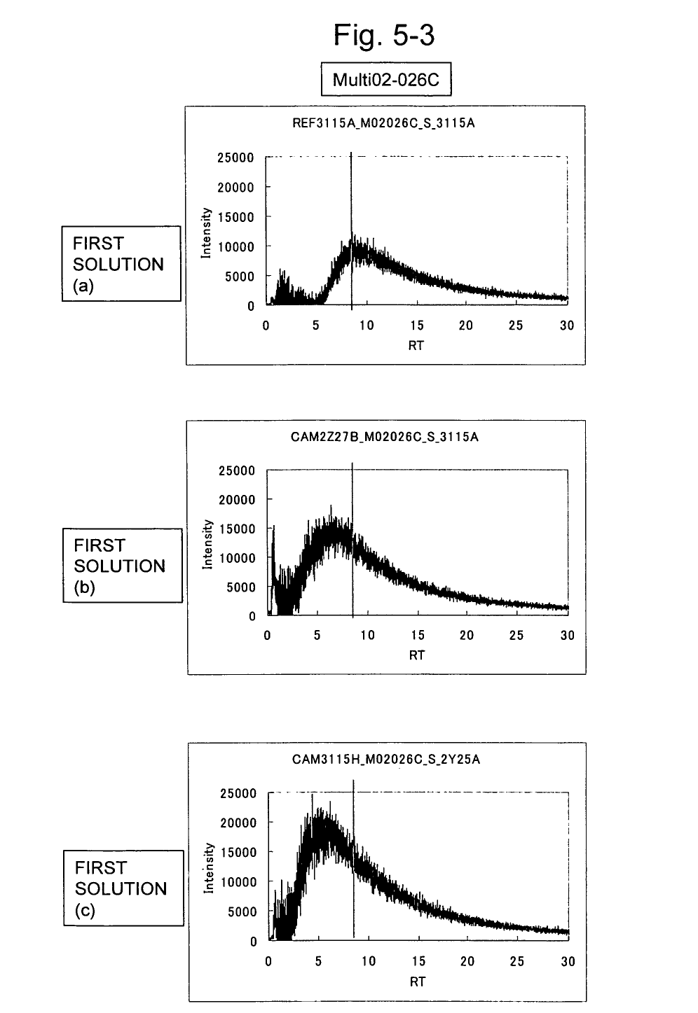

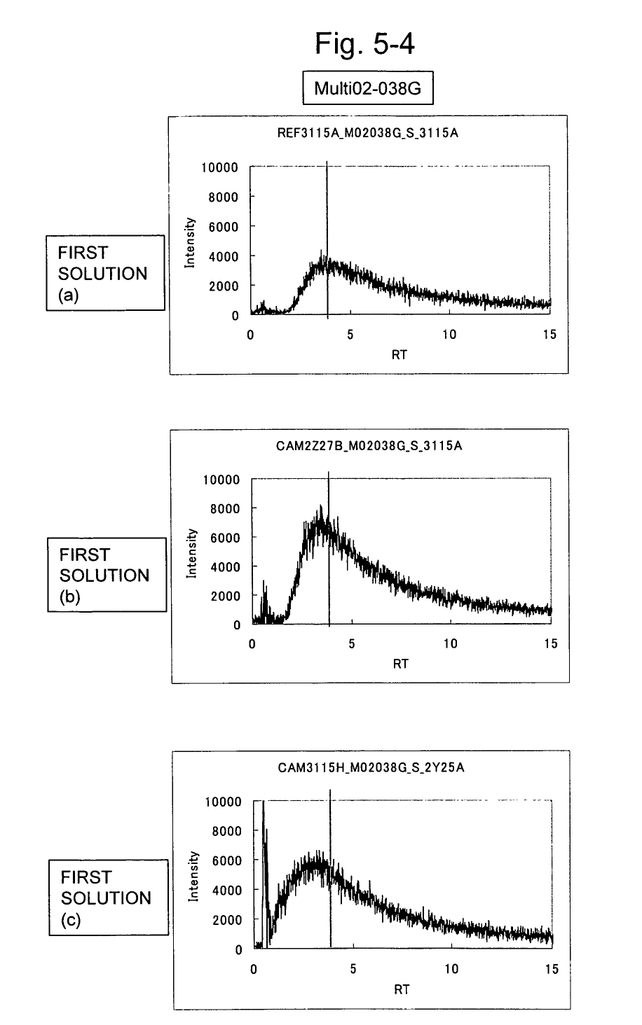

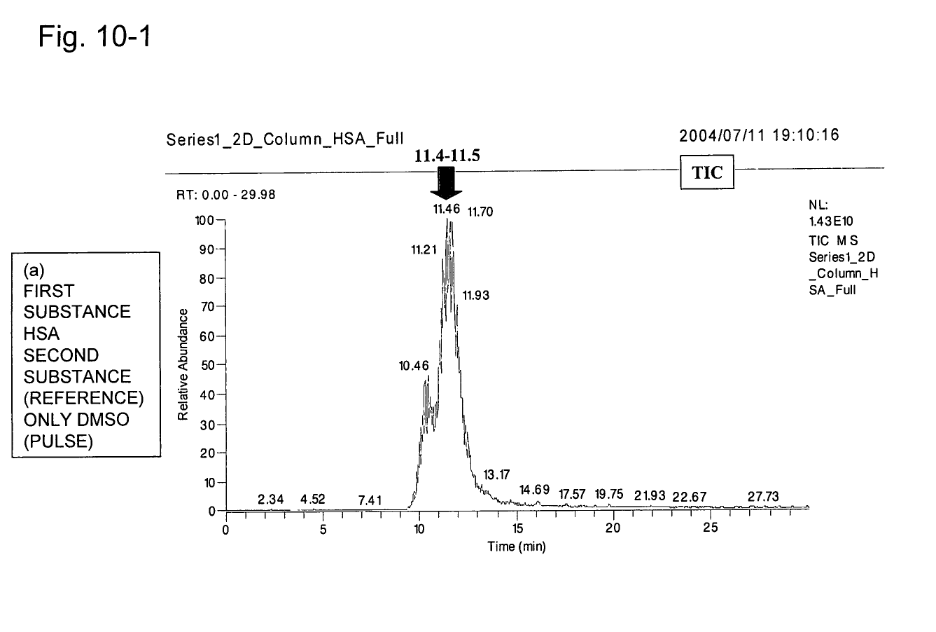

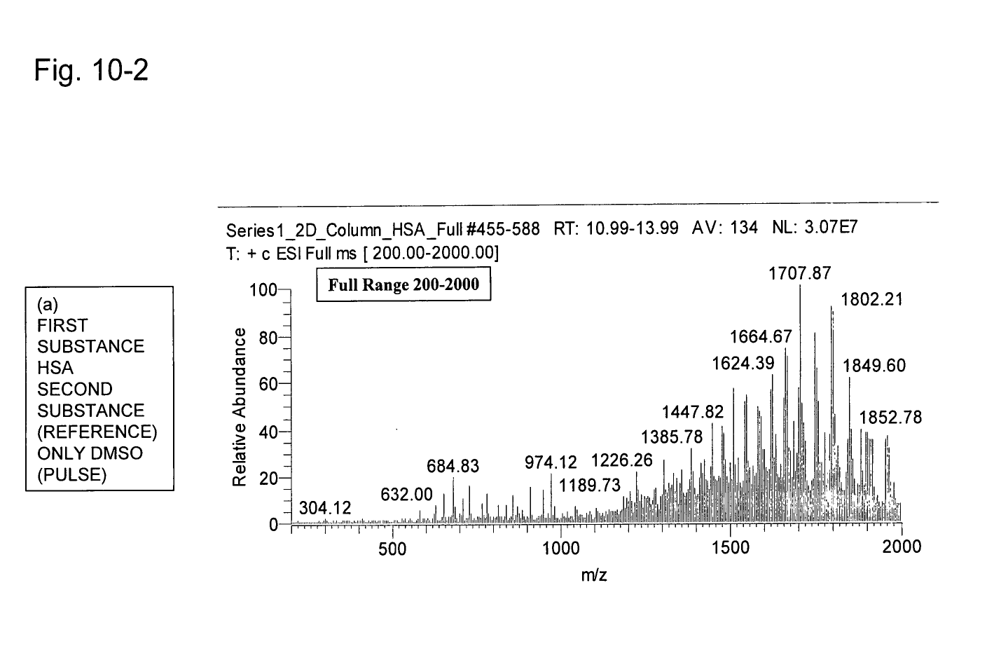

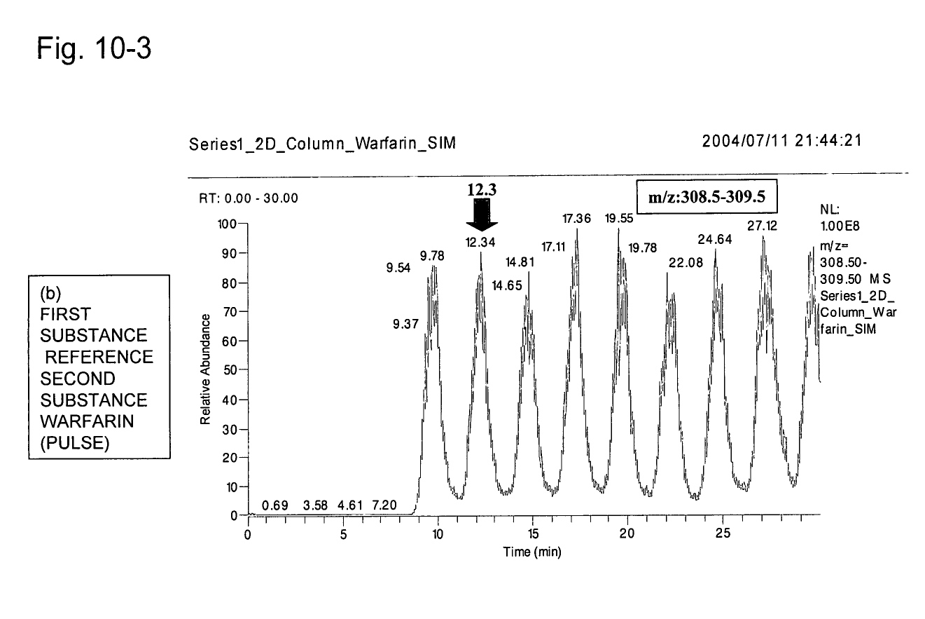

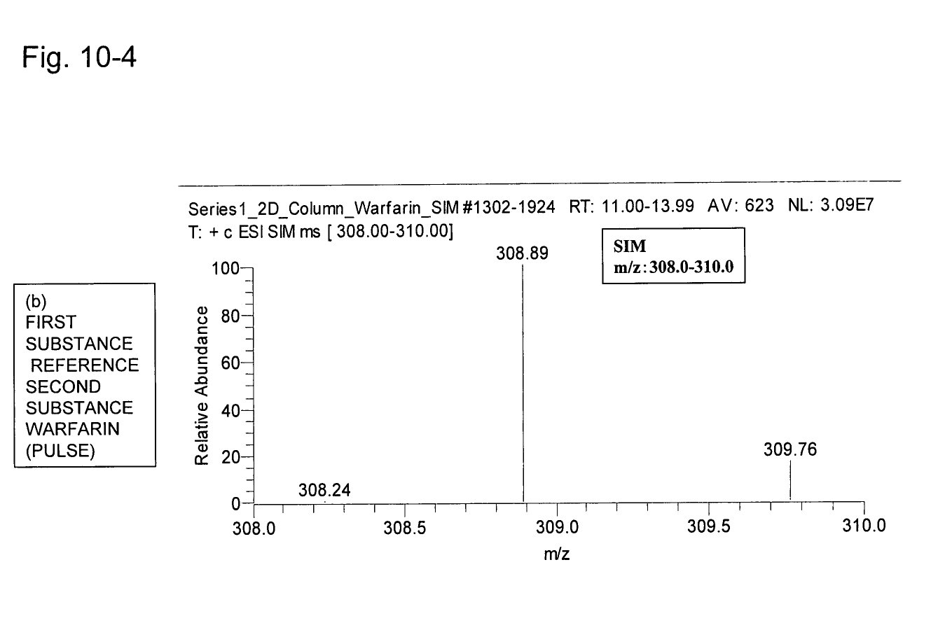

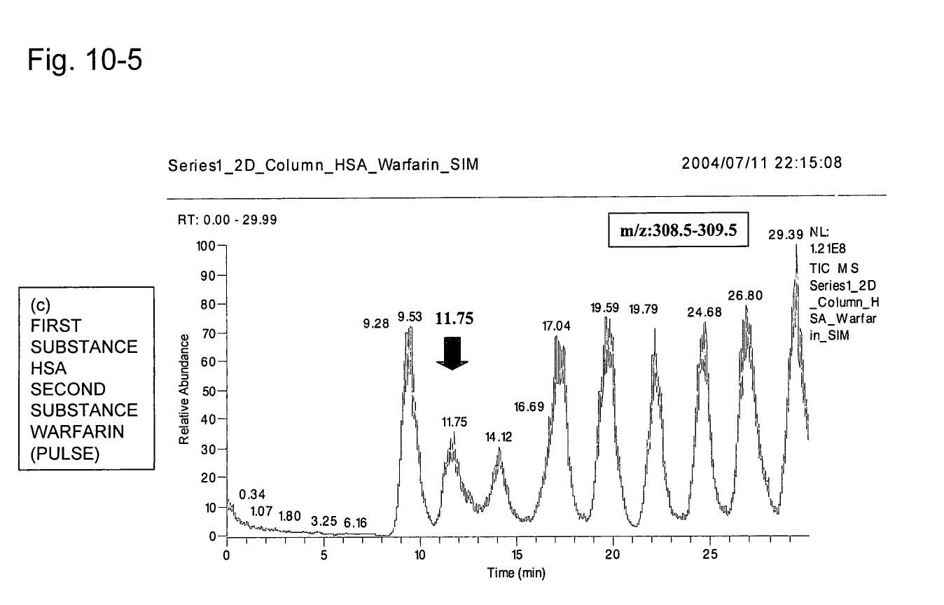

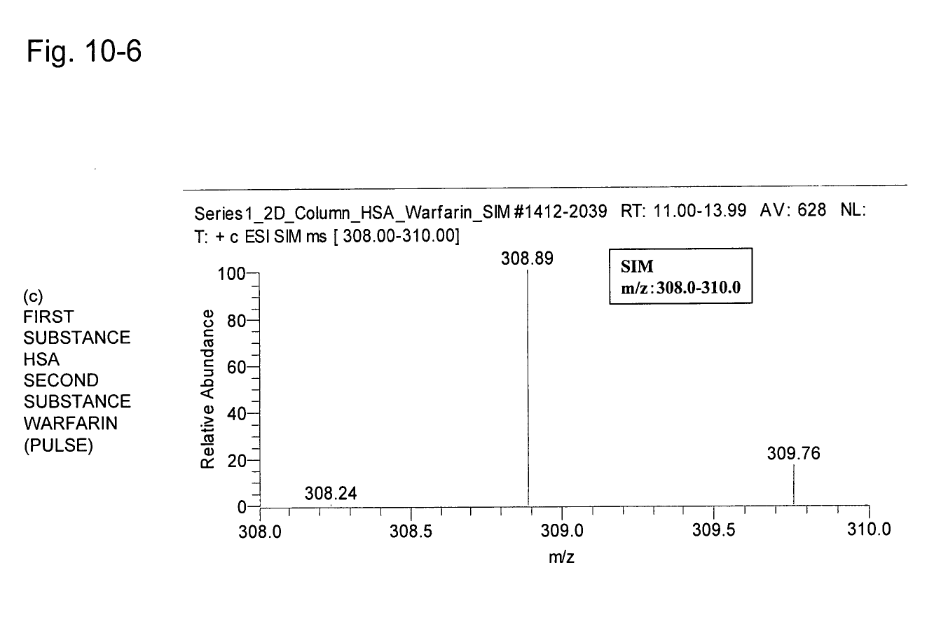

When the first injector injected a first solution which did not contain the first substance, a mass chromatogram of Warfarin was detected in the similar pulses at timed intervals (FIG. 10-3). However, when the first injector injected a first solution which contained the first substance HSA, the elution peaks which correspond to the peaks at 12.3 min and 14.8 min in Measurement 4 (b) appeared earlier at 11.8 min and 14.1 min respectively (FIG. 10-5). HSA itself was eluted from the 2^{nd} column at 11.5 min (FIG. 10-1) which corresponds to the elution time of the Warfarin pulse modulation which was eluted earlier in FIG. 10-5. This means that HSA which was eluted from the 1^{st} stage column passed over the Warfarin pulses at 12.3 min and 14.8 min in FIG. 10-3 in the 2^{nd} stage column so that the Warfarin pulses were eluted earlier at 11.8 min and 14.1 min in FIG. 10-5. This shows that a change in a mass chromatogram of the second substance Warfarin at the 2^{nd} stage column made it possible to determine whether an elute in the 1^{st} stage column contained a first substance to interact with Warfarin, and at which elution time the first substance was eluted from the 1^{st} stage column.

The publications, patents, and patent applications referenced herein are incorporated herein by reference in their entireties.

1. A method for analyzing interactions, comprising the steps of:
introducing into a separation channel a first solution comprising a substance to be analyzed that is eluted from the separation channel faster, and a second solution comprising a substance to be analyzed that is eluted from the separation channel more slowly, wherein at least a portion of the first solution is introduced into the separation channel after introducing at least a portion of the second solution thereinto; and
detecting a chromatogram of the substances eluted from the separation channel.

2. The method for analyzing interactions according to claim 1, further comprising the step of comparing the detected chromatogram with a chromatogram of the substance comprised in the first solution and/or the substance comprised in the second solution without any interaction with other substances to be analyzed; and
wherein a determination that there exists an interaction between the substance comprised in the first solution and the substance comprised in the second solution is made, when there is a difference between the chromatograms.

3. The method for analyzing interactions according to claim 1, wherein the separation channel is composed of at least one chromatography selected from the group consisting of size exclusion chromatography, ion exchange chromatography, affinity chromatography, adsorption chromatography, hydrophobic chromatography, hydroxyapatite chromatography, metal chelate chromatography, an electrophoresis tube and an electroosmotic flow tube.

4. The method for analyzing interactions according to claim 1, wherein the chromatogram is detected by at least one detector selected from the group consisting of a mass spectrometry detector, a spectroscopy detector, a UV detector, a fluorescence detector, a luminescence detector, a refraction detector, and an electrochemical detector.

5. The method for analyzing interactions according to claim 1, wherein the first solution and/or the second solution comprise a plurality of substances to be analyzed.

6. The method for analyzing interactions according to claim 1, wherein the chromatogram is a mass chromatogram detected based on the mass of a substance to be analyzed comprised in the first solution and/or the second solution.

7. The method for analyzing interactions according to claim 1, wherein the first solution and/or the second solution comprise a plurality of substances to be analyzed, and a multiplex chromatogram of the plurality of the substances are detected.

8. The method for analyzing interactions according to claim 1, wherein the first solution and the second solution are introduced into the separation channel in different amounts.

9. The method for analyzing interactions according to claim 1, wherein the second solution is introduced into the separation channel in an amount twice or more the amount of the first solution.

10. The method for analyzing interactions according to claim 1, wherein the step of introducing at least a portion of the first solution into the separation channel after introducing at least a portion of the second solution thereinto comprises introducing a gaseous or liquid spatial sample after the introduction of the second solution and before the introduction of the first solution.

11. The method for analyzing interactions according to claim 1, wherein the first solution and/or the second solution consist of a plurality of solution samples, and the plurality of solution samples are introduced continuously.

12. The method for analyzing interactions according to claim 1, wherein the separation channel consists of n stages (n≧2, integer), and a step of introducing a fraction eluted from an (m−1)^{th} stage (2≦m≦n, integer) of the separation channel into an m^{th} stage of the separation channel is repeated from m=2 until m=n, and
the step of detecting a chromatogram comprises detecting a chromatogram of a substance to be analyzed eluted from an n^{th} stage of the separation channel.

13. The method for analyzing interactions according to claim 12, wherein
when the fraction eluted from the (m−1)^{th} stage of the separation channel contains the substance comprised in the first solution, the fraction is introduced into the m^{th} stage of the separation channel after the introduction of the second solution, and
when the fraction eluted from the (m−1)^{th} stage of the separation channel contains the substance comprised in the second solution, the fraction is introduced into the m^{th} stage of the separation channel before the introduction of the first solution.

14. The method for analyzing interactions according to claim 12, wherein
when the fractions eluted from the (m−1)^{th} stage of the separation channel contain the substance comprised in the first solution, the fractions are introduced into the m^{th} stage of the separation channel during the second solution is introduced into the m^{th} stage of the separation channel at predetermined intervals, and
when the fractions eluted from the (m−1)^{th} stage of the separation channel contain the substance comprised in the second solution, the fractions are introduced into the m^{th} stage of the separation channel during the first solution is introduced into the m^{th} stage of the separation channel at predetermined intervals.

15. The method for analyzing interactions according to claim 1, wherein the step of introducing at least a portion of the first solution into the separation channel after introducing at least a portion of the second solution thereinto comprises introducing the first solution and the second solution in an amount of 10 μL or less respectively.

16. The method for analyzing interactions according to claim 1, wherein the step of introducing at least a portion of the first solution into the separation channel after introducing at least a portion of the second solution thereinto comprises introducing the first solution and the second solution into an amount of 3 μL or less respectively.

17. An apparatus for analyzing interactions, wherein the apparatus comprises:
a separation device which has a separation channel to separate and elute substances comprised in a solution;
a container section which has first solutions comprising substances that are eluted from the separation channel faster and second solutions comprising substances that are eluted from the separation channel more slowly;
an introduction device to introduce at least a portion of the first solution from the container section into the separation channel after introducing at least a portion of the second solution thereinto; and
a control device to control operation of at least the introduction device,
wherein the control device controls the introduction device to introduce the second solution and the first solution into the separation channel in this order.

18. The apparatus for analyzing interactions according to claim 17, wherein the apparatus further comprises a detection device to detect chromatograms of the substances eluted from the separation channel.

19. The apparatus for analyzing interactions according to claim 17, wherein the separation device has at least one chromatography selected from the group consisting of a size exclusion chromatography, an ion exchange chromatography, an affinity chromatography, an adsorption chromatography, a hydrophobic chromatography, a hydroxyapatite chromatography, a metal chelate chromatography, an electrophoresis tube device, and an electroosmotic flow tube device.

20. The apparatus for analyzing interactions according to claim 18, wherein the detection device is at least one detector selected from the group consisting of a mass spectrometry detector, a spectroscopy detector, a UV detector, a fluorescence detector, a luminescence detector, a refraction detector, and an electrochemical detector.

21. The apparatus for analyzing interactions according to claim 17, wherein the control device controls the introduction device to introduce a gaseous or liquid spatial sample after the introduction of the second solution and before the introduction of the first solution.

22. The apparatus for analyzing interactions according to claim 17, wherein the container section has a plurality of first solutions and/or a plurality of second solutions.

23. The apparatus for analyzing interactions according to claim 17, wherein
the separation device comprises separation channels consisting of n stages (n≧2, integer), and
the control device controls the introduction device so that a step of introducing fractions eluted from an (m−1)^{th} stage (2≦m≦n, integer) of the separation channel into an m^{th} stage of the separation channel is repeated from m=2 until m=n.

24. The apparatus for analyzing interactions according to claim 23, wherein the control device controls the introduction device so that when the fractions eluted from the (m−1)^{th} stage of the separation channel contain the substance comprised in the first solution, the fractions are introduced into the m^{th} stage of the separation channel after the introduction of the second solution, and when the fractions eluted from the (m−1)^{th} stage of the separation channel contain the substance comprised in the second solution, the fractions are introduced into the m^{th} stage of the separation channel before the introduction of the first solution.

25. The apparatus for analyzing interactions according to claim 23, wherein the control device controls the introduction device so that when the fractions eluted from the (m−1)^{th} stage of the separation channel contain the substance comprised in the first solution, the fractions are introduced into the m^{th} stage of the separation channel during the second solution is introduced into the m^{th} stage of the separation channel at predetermined intervals, and
when the fractions eluted from the (m−1)^{th} stage of the separation channel contain the substance comprised in the second solution, the fractions are introduced into the m^{th} stage of the separation channel during the first solution is introduced into the m^{th} stage of the separation channel at predetermined intervals.

26. The apparatus for analyzing interactions according to claim 17, wherein the first solution and the second solution are introduced into an amount of 10 μL or less respectively.

27. The apparatus for analyzing interactions according to claim 17, wherein the first solution and the second solution are introduced into an amount of 3 μL or less respectively.
